(19) United States
(12) Patent Application Publication
(10) Pub. No.: US 2025/0161505 A1
(43) Pub. Date: May 22, 2025

(54) CD206 Targeted Peptide Conjugates and Methods of Using the Same

(71) Applicants: Riptide Bioscience, Inc., Vallejo, CA (US); The UAB Research Foundation, Birmingham, AL (US)

(72) Inventors: Jesse M. Jaynes, Auburn, AL (US); Clayton Yates, Auburn, AL (US); Henry W. Lopez, Napa, CA (US); Candace C. Parker, Tuscaloosa, AL (US); Suzanne E. Lapi, Tuscaloosa, AL (US)

(21) Appl. No.: 18/837,309

(22) PCT Filed: Feb. 14, 2023
(86) PCT No.: PCT/US2023/013028

§ 371 Date: Aug. 9, 2024

Related U.S. Application Data
(60) Provisional application No. 63/309,787, filed on Feb. 14, 2022.

(51) Int. Cl. A61K 51/08; G01N 33/569; G01N 33/60

(52) U.S. Cl. CPC A61K 51/088; G01N 33/56972; G01N 33/60; A61K 2121/00; A61K 2123/00; G01N 2333/70596

(57) ABSTRACT

CD206 targeted peptide conjugates are provided. Aspects of the conjugates include a CD206 binding peptide conjugated to a label or therapeutic agent. Also provided are methods of using the conjugates, e.g., in diagnostic or therapeutic applications.

### CROSS-REFERENCE TO RELATED APPLICATION

Pursuant to 35 U.S.C. § 119 (e), this application claims priority to the filing date of U.S. Provisional Patent Application Ser. No. 63/309,787 filed Feb. 14, 2022; the disclosure of which application is herein incorporated by reference.

### INTRODUCTION

Tumor associated macrophages (TAMs) are large phagocytic cells that play numerous roles in cancer biology and are an important component of the relationship between immune system response and tumor progression.¹ It has been demonstrated that TAMs influence chemoresistance, immune regulation, tumor initiation, and tumor growth, both directly and indirectly.^{2,3} Macrophages can be loosely categorized into two core groups with differing roles in immune defense and immune surveillance: classically activated macrophages (M1) and alternatively activated macrophages (M2).^{1,4} M1 macrophages are considered anti-tumor due to their ability to kill tumor cells through the production of pro-inflammatory cytokines and are the more prominent phenotype present in the earlier stages of cancer. M2 macrophages are considered protumor due to their production of anti-inflammatory cytokines and are more common as the cancer progresses.^{5, 6}

The macrophage mannose receptor (CD206) is a C-type lectin that is expressed on M2 macrophages. CD206 represents a surface receptor on macrophages that operates as a pattern recognition receptor of pathogens such as viruses, fungi, and bacteria.⁷ Consequently, CD206 is involved in the recognition of immune pathogens following antigen internalization and presentation.⁸

RP-832c is a highly specific CD206 targeted peptide identified from in-silico biophysical homology screening and is cross reactive to both human and murine CD206.⁹ It is classified among innate defense regulators that are synthetic peptide analogs of naturally occurring antimicrobial peptides involved in aspects of the innate immune system.¹⁰ RP-832c has also shown the capability to shift the population of tumor associated macrophages (TAMs) from M2 (pro-tumor) towards a M1 phenotype (anti-tumor) and has shown promise in inhibiting tumor resistance in PDL1 unresponsive melanoma murine models. Additionally, RP-832c has been demonstrated to inhibit bleomycin induced pulmonary fibrosis through interactions with CD206 macrophages.^{9, 11}

Molecular imaging techniques can aid in identifying biomarkers of disease, tracking disease progression, and monitoring therapeutic responses. Positron emission tomography (PET) is a form of molecular imaging that allows for a noninvasive assessment of physiologic information by utilizing radioactive contrast agents. It is widely used in both clinical and preclinical settings due to its translational possibilities, sensitivity, and quantitative accuracy. The use of PET has proved to be an instrumental tool in the diagnosis and monitoring of a variety of disease states including neurological and cardiovascular indications and cancer.¹²

### SUMMARY

CD206 targeted peptide conjugates are provided. Aspects of the conjugates include a CD206 binding peptide conjugated to a label or therapeutic agent. Also provided are methods of using the conjugates, e.g., in diagnostic or therapeutic applications.

### BRIEF DESCRIPTION OF THE FIGURES

FIG. 1: Radiosynthesis of [⁶⁸Ga]RP832c

FIG. 2 demonstrates binding studies for [⁶⁸Ga]RP832c and [⁶⁸Ga]MART-1 to mouse CD206 protein.

FIGS. 3A-3C provide the results of surface plasmon resonance experiments, as described in the Experimental Section below.

FIGS. 4A-4D: Representative fused dynamic PET/CT images of CT26 tumor bearing mice injected with [⁶⁸Ga]RP832c (A. MIP view; C. axial view) and [⁶⁸Ga]MART-1 ((B. MIP view; D. axial view). Color scale shows PET image from integrated data 10-15 minutes, 25-30 minutes, and 40-45 minutes after injection. White circles show tumor location.

FIGS. 5A-5D: Representative fused dynamic PET/CT images of 4T1 tumor bearing mice injected with [⁶⁸Ga]RP832c (A. MIP view; C. axial view) and [⁶⁸Ga]MART-1 ((B. MIP view; D. axial view). Color scale shows PET image from integrated data 10-15 minutes, 25-30 minutes, and 40-45 minutes after injection. White circles show tumor location.

FIG. 6: Tumor time activity curves for A) CT26 and B) 4T1 tumor bearing mice injected with [⁶⁸Ga]RP832c and [⁶⁸Ga]MART-1.

FIGS. 7A-7C: A) Biodistribution of [⁶⁸Ga]RP832c in CT26 and 4T1 tumor bearing mice 1.5 hours after injection. Immunohistochemical staining for CD206 in B) CT26 and C) 4T1 tumor tissue.

### DEFINITIONS

In describing and claiming the invention, the following terminology will be used in accordance with the definitions set forth below.

As use herein, the terms “administration of” and or “administering” a compound should be understood to mean providing a compound of the invention or a prodrug of a compound of the invention to a subject in need of diagnosis or treatment. As used herein, “alleviating a disease or disorder symptom,” means reducing the severity of the symptom or the frequency with which such a symptom is experienced by a patient, or both.

The term “cancer”, as used herein, is defined as proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer, and lung cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “chemically conjugated,” or “conjugating chemically” refers to linking two entities together, e.g., a CD206 targeting peptide and a label or therapeutic agent. This linking also includes covalent bonds created between the two entities using chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the two entities.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups: I. Small aliphatic, nonpolar or slightly polar residues: Ala, Ser, Thr, Pro, Gly; II. Polar, negatively charged residues and their amides: Asp, Asn, Glu, Gln; III. Polar, positively charged residues: His, Arg, Lys; IV. Large, aliphatic, nonpolar residues: Met Leu, Ile, Val, Cys; V. Large, aromatic residues: Phe, Tyr, Trp.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

The term “elixir,” as used herein, refers in general to a clear, sweetened, alcohol-containing, usually hydroalcoholic liquid containing flavoring substances and sometimes active medicinal agents.

As used herein “injecting or applying” includes administration of a compound of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, the term “invasive,” or “metastasis” as used herein, refers to any migration of cells, especially to invasive cancer cells or tumor cells. The term applies to normally invasive cells such as wound-healing fibroblasts and also to cells that migrate abnormally. Although the term is not to be limited by any mechanistic rationale, such cells are thought to migrate by defeating the body's means for keeping them sufficiently “in place” to function normally. Such cells are “invasive” if they migrate abnormally within a tissue or tumor, or escape the tissue, or invade other tissues.

As used herein, the term “malignant” refers to having the properties of anaplasia, penetrance, such as into nearby areas or the vasculature, and metastasis.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

As used herein, the term “post surgical tumor tissue” refers to cancerous tissue (e.g., biopsy tissue) that has been removed from a subject (e.g., during surgery).

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

A “prophylactic” treatment is a treatment administered to a subject who does not exhibit signs of a disease or exhibits only early signs of the disease for the purpose of decreasing the risk of developing pathology associated with the disease.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. As used herein, the term “non-cancerous” in reference to a pancreatic cell refers to a cell demonstrating regulatable cell growth and functional physiology relative to its developmental stage and activity.

As used herein, a “subject in need thereof” is a patient, animal, mammal, or human, who will benefit from the method of this invention.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “tumor” refers to an abnormal mass of tissue that results from excessive cell division that is uncontrolled and progressive. It is also called a neoplasm. Tumors may be either benign (not cancerous) or malignant.

As used herein, the term “tumor cell”, as used herein, refers to any mass of cells that exhibits any uncontrolled growth patterns or altered physiology. Tumor cells may be derived from any tissue within an organism (e.g., a pancreatic ductal tumor cell). As used herein, the term “cancer” is a general term for more than 100 diseases that are characterized by an uncontrolled, abnormal growth of cells. Cancer cells can spread locally or can intravasate and spread via the bloodstream and lymphatic system to other parts of the body and form metastases. Cancer cells that spread are called “malignant.” As used herein, the terms “cancer” and “cancerous” in reference to a physiological condition in mammals is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

### DETAILED DESCRIPTION

CD206 targeted peptide conjugates are provided. Aspects of the conjugates include a CD206 binding peptide conjugated to a label or therapeutic agent. Also provided are methods of using the conjugates, e.g., in diagnostic or therapeutic applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, CD206 targeted peptide conjugates are provided. Aspects of the conjugates include a CD206 binding peptide conjugated to a label or therapeutic agent.

### CD206 Targeted Peptide Conjugates

As summarized above, aspects of the invention include CD206 targeted peptide conjugates. CD206 targeted peptide conjugates include a CD206 binding peptide conjugated to, i.e., linked or bonded to, either directly or via a linking group, to one or more payload entities, which payload may vary and in some instances is a label or therapeutic agent. The CD206-binding peptide may be conjugated to the payload such as by hydrogen bonding or ionic interactions. In other embodiments, the CD206-binding peptide is conjugated to the payload with one or more covalent bonds. The CD206-binding peptide may be directly bonded to the payload or may be bonded to the payload through one or more linkers, where in certain instances, the CD206-binding peptide and the active agent are bonded by linking chemistry that includes but is not limited to, maleimide/thiol, succimidylester (NHS ester)/amine, azide chemistry, carboxy/EDC (1-Ethyl-3-[3-dimethylaminopropyl]carbodiimide Hydrochloride)/amine, amine/Sulfo-SMCC (Sulfosuccinimidyl 4-[N-maleimidomethyl]cyclohexane-1-carboxylate)/thiol, and amine/BMPH (N-[β-Maleimidopropionic acid]hydrazide.TFA)/thiol. The various components of the CD206 targeted peptide conjugates are now described further in greater detail.

### CD206 Binding Peptide

Conjugates of embodiments of the invention include a CD206 binding peptide. The terms “peptide” and “polypeptide” are used synonymously herein to refer to polymers constructed from amino acid residues. The term “amino acid residue,” as used herein, refers to any naturally occurring amino acid, non-naturally occurring amino acid, or amino acid mimetic (such as a peptoid monomer). An amino acid residue can be in an L- or D-form. CD206 binding peptides of interest include, but are not limited to, those described in U.S. Pat. Nos. 9,492,499 and 10,413,584; the disclosures of which are herein incorporated by reference.

CD206 binding peptides may vary in length. The “length” of a CD206 binding peptide is the number of amino acid residues linked end-to-end that constitute the polypeptide, excluding any non-peptide linkers and/or modifications that the polypeptide may contain. In some embodiments, the peptide is of 5 to 30 amino acid residues (e.g., 5 to 25, 10 to 20 or 5 to 18, 5 to 12 or 5 to 10, or 6 to 30, 6 to 25, 6 to 20, 6 to 18, 6 to 12, 6 to 10 or 7 to 12, or 7 to 10 amino acid residues) in length. In some instances, the peptide includes an amphipathic region, i.e., a region having both hydrophilic and hydrophobic parts. In some instances, the peptide comprises an alternating sequence of hydrophobic and hydrophilic modules. A “hydrophobic module” is made up of a peptide sequence consisting of one to five hydrophobic amino acid residues. Likewise, a hydrophilic module is made up of a peptide sequence consisting of one to five hydrophilic amino acid residues. In some embodiments, the peptide is of 5 to 12 amino acid residues (e.g., 6, 7, 8, 9 or 10 amino acid residues) in length, and comprises a region of alternating hydrophilic and hydrophobic modules that adopts an amphipathic conformation under physiological conditions. In certain instances, a region of alternating hydrophilic and hydrophobic modules of the peptide is of 5 to 18 amino acid residues in length (e.g., 6 to 18, 6 to 14, 6 to 12, 7 to 12, or 5, 6, 7, 8, 9, 10, 11 or 12 amino acids in length), wherein the peptide is optionally further modified (e.g., as described herein). Such a region, when present, can comprise: 2 or more (e.g., 3 or more or 4 or more) hydrophobic modules; and one or more (e.g., 2 or more, 3 or more, or 4 or more) hydrophilic modules (e.g., each comprising at least one cationic residue). In some embodiments, the subject immunomodulatory peptides (e.g., as described herein) are CD206-binding peptides. In some instances, such a region, when present, of the peptide has a length of 6 to 12 amino acid residues, such as 7 to 12. In some instances, the such a region, when present, of the peptide has a length of 6 to 10 amino acid residues. The hydrophobic modules can consist of any convenient residues. In certain instances, the hydrophobic modules include amino acid residues selected from phenylalanine, tryptophan, alanine, valine, and glycine. The binding peptide can comprise 2, 3 or more hydrophilic modules that consist of any convenient residues. In some instances, the hydrophilic modules include amino acid residues selected from lysine, arginine, histidine, aspartic acid, glutamic acid, asparagine and glutamine.

In some instances, the CD206 binding peptide is a Class II peptide. Class II peptides can comprise, consist essentially of, or consist of a region of alternating hydrophilic and hydrophobic modules, e.g., as described, above, that includes a sequence selected from the group of sequences defined by Formula I:

Y₁ₐ-X₁ₐ-X_{1b}-Y₂ₐ-Y_{2b}-X₂ₐ-X_{2b}-Y₃ₐ (Formula I).

Amino acid residue Y₂ₐ in Formula I can be selected from the group consisting of Phe (F), Trp (W), Tyr (Y), Leu (L), Cys (C), Met (M), Val (V), Ile (I), Pro (P), Thr (T), Ser(S), Ala (A), and Gly (G). In certain embodiments, amino acid residue Y₂ₐ in Formula I is selected from the group consisting of Phe (F), Trp (W), and Tyr (Y). Alternatively, amino acid residue Y₂ₐ in Formula I can be selected from the group consisting of Leu (L), Cys (C), Met (M), Val (V), Ile (I). Amino acid residue Y_{2b} in Formula I can be selected from the group consisting of Phe (F), Trp (W), Tyr (Y), Leu (L), Cys (C), Met (M), Val (V), Ile (I), Pro (P), Thr (T), Ser(S), Ala (A), and Gly (G). In certain embodiments, amino acid residue Y_{2b} in Formula I is selected from the group consisting of Phe (F), Trp (W), and Tyr (Y). Alternatively, amino acid residue Y_{2b} in Formula I can be selected from the group consisting of Leu (L), Cys (C), Met (M), Val (V), Ile (I). Amino acid residue X_{1b} in Formula I can be selected from the group consisting of Arg (R), Lys (K), and His (H). Alternatively amino acid residue X_{1b} in Formula I can be selected from the group consisting of Asn (N), Gln (Q), Asp (D), and Glu (E). Amino acid residue X₂ₐ in Formula I can be selected from the group consisting of Arg (R), Lys (K), and His (H). Alternatively, amino acid residue X₂ₐ can be selected from the group consisting of Asn (N), Gln (Q), Asp (D), and Glu (E). The sequence X_{1b}-Y₂ₐ-Y_{2b}-X₂ₐ in Formula I can be selected from the group consisting of Lys-Phe-Phe-Lys (KFFK; SEQ ID NO: 01), Lys-Trp-Trp-Lys (KWWK; SEQ ID NO: 02), Lys-Tyr-Try-Lys (KYYK; SEQ ID NO: 03), Lys-Phe-Trp-Lys (KFWK; SEQ ID NO: 04), Lys-Trp-Phe-Lys (KWFK; SEQ ID NO: 05), Lys-Phe-Tyr-Lys (KFYK; SEQ ID NO: 06), Lys-Tyr-Phe-Lys (KYFK; SEQ ID NO: 07), Lys-Trp-Tyr-Lys (KWYK; SEQ

ID NO: 08), and Lys-Tyr-Trp-Lys (KYWK; SEQ ID NO: 09). Alternatively, the sequence X_{1b}-Y₂ₐ-Y_{2b}-X₂ₐ in Formula I can be selected from the group consisting of Arg-Phe-Phe-Arg (RFFR; SEQ ID NO: 10), Arg-Trp-Trp-Arg (RWWR; SEQ ID NO: 11), Arg-Tyr-Try-Arg (RYYR; SEQ ID NO: 12), Arg-Phe-Trp-Arg (RFWR; SEQ ID NO: 13), Arg-Trp-Phe-Arg (RWFR; SEQ ID NO: 14), Arg-Phe-Tyr-Arg (RFYR; SEQ ID NO: 15), Arg-Tyr-Phe-Arg (RYFR; SEQ ID NO: 16), Arg-Trp-Tyr-Arg (RWYR; SEQ ID NO: 17), and Arg-Tyr-Trp-Arg (RYWR; SEQ ID NO: 18). In other alternatives, the sequence X_{1b}-Y₂ₐ-Y_{2b}-X₂ₐ in Formula I can be selected from the group consisting of His-Phe-Phe-His (HFFH; SEQ ID NO: 19), His-Trp-Trp-His (HWWH; SEQ ID NO: 20), His-Tyr-Try-His (HYYH; SEQ ID NO: 21), His-Phe-Trp-His (HFWH; SEQ ID NO: 21), His-Trp-Phe-His (HWFH; SEQ ID NO: 23), His-Phe-Tyr-His (HFYH; SEQ ID NO: 24), His-Tyr-Phe-His (HYFH; SEQ ID NO: 25), His-Trp-Tyr-His (HWYH; SEQ ID NO: 26), and His-Tyr-Trp-His (HYWH; SEQ ID NO: 27). Amino acid residue X₁ₐ in Formula I can be selected from the group consisting of Arg (R), Lys (K), His (H), Asn (N), Gln (Q), Asp (D), and Glu (E). In certain embodiments, amino acid residue X₁ₐ is selected from the group consisting of Arg (R) and Gln (Q). In certain embodiments, amino acid residue X₁ₐ in Formula I is Arg (R). Alternatively, amino acid residue X₁ₐ in Formula I can be selected from the group consisting of Lys (K), Gln (Q), Glu (E), and Asn (N).

Amino acid residue X_{2b} in Formula I can be selected from the group consisting of Arg (R), Lys (K), His (H), Asn (N), Gln (Q), Asp (D), and Glu (E). In certain embodiments, amino acid residue X_{2b} is selected from the group consisting of Arg (R) and Gln (Q). In certain embodiments, amino acid residue X_{2b} in Formula I is Arg (R). Alternatively, amino acid residue X_{2b} in Formula I can be selected from the group consisting of Lys (K), Gln (Q), Glu (E), and Asn (N). Amino acid residue Y₁ₐ in Formula I can be selected from the group consisting of Phe (F), Trp (W), Tyr (Y), Leu (L), Cys (C), Met (M), Val (V), Ile (I), Thr (T), Pro (P), Ser(S), Ala (A), and Gly (G). In certain embodiments, amino acid residue Y₁ₐ in Formula I is selected from the group consisting of Phe (F), Trp (W), and Tyr (Y). Alternatively, amino acid residue Y₁ₐ in Formula I can be selected from the group consisting of Leu (L), Cys (C), Met (M), Val (V), Ile (I). Amino acid residue Y₃ₐ in Formula I can be selected from the group consisting of Phe (F), Trp (W), Tyr (Y), Leu (L), Cys (C), Met (M), Val (V), Ile (I), Thr (T), Pro (P), Ser(S), Ala (A), and Gly (G). In certain embodiments, amino acid residue Y₃ₐ in Formula I is selected from the group consisting of Phe (F), Trp (W), and Tyr (Y). Alternatively, amino acid residue Y₃ₐ in Formula I can be selected from the group consisting of Leu (L), Cys (C), Met (M), Val (V), Ile (I).

A Class II peptide can comprise, consist essentially of, or consist of a region of alternating hydrophilic and hydrophobic modules that further includes a first additional amino acid residue directly bound to amino acid residue Y3a of Formula I. The first additional amino acid residue can be a hydrophobic amino acid residue (e.g., a residue selected from the group consisting of Phe (F), Trp (W), Tyr (Y), Leu (L), Cys (C), Met (M), Val (V), Ile (I), Thr (T), Pro (P), Ser(S), Ala (A), and Gly (G); a residue selected from the group consisting of Phe (F), Trp (W), and Tyr (Y); a residue selected from the group consisting of Phe (F), Trp (W), Tyr (Y), and Leu (L); or, a residue selected from the group consisting of Leu (L), Cys (C), Met (M), Val (V), and Ile (I). Alternatively, the first additional amino acid residue can be a hydrophilic amino acid residue (e.g., a residue selected from the group consisting of Arg (R), Lys (K), His (H), Asn (N), Gln (Q), Asp (D), and Glu (E); a residue selected from the group consisting of Arg (R), Lys (K), and His (H); a residue selected from the group consisting Arg (R), Lys (K), His (H), and Gln (Q); or a residue selected from the group consisting of Asn (N), Gln (Q), Asp (D), and Glu (E)).

A Class II peptide can comprise, consist essentially of, or consist of a region of alternating hydrophilic and hydrophobic modules that further includes a first additional amino acid residue directly bound to amino acid residue Y₁ₐ of Formula I and a second additional amino acid reside directly bound to amino acid residue Y₃ₐ of Formula I. The first additional amino acid residue can be a hydrophobic amino acid residue and the second additional amino acid residue can be a hydrophilic amino acid residue. Alternatively, the first additional amino acid residue can be a hydrophilic amino acid residue and the second amino acid residue can be a hydrophobic amino acid residue. Regardless, the additional hydrophobic amino acid residue can be selected from the group consisting of Phe (F), Trp (W), Tyr (Y), Leu (L), Cys (C), Met (M), Val (V), Ile (I), Thr (T), Pro (P), Ser (S), Ala (A), and Gly (G); and in certain embodiments from the group consisting of Phe (F), Trp (W), and Tyr (Y); and in additional embodiments from the group consisting of Phe (F). The additional hydrophilic amino acid residue can be selected from the group consisting of Arg (R), Lys (K), His (H), Asn (N), Gln (Q), Asp (D), and Glu (E); and in certain embodiments, a residue selected from the group consisting of Arg (R), Lys (K), and His (H); or a residue selected from the group consisting of Asn (N), Gln (Q), Asp (D), and Glu (E).

A Class II peptide can comprise, consist essentially of, or consist of a region of alternating hydrophilic and hydrophobic modules comprising, consisting essentially of, or consisting of a sequence selected from the group of sequences listed in Table 1:

TABLE 1
Class II peptides

| | | SEQ ID |
|---|---|---|
| RP# | Sequence | NO: |
| 124 | FFQKFFKRWR | 28 |
| 132 | FFRKFFKRFR | 29 |
| 134 | RFRKFFKRFF | 30 |
| 142 | RFRKFFKQFF | 31 |
| 147 | FFQKFFKRFR | 32 |
| 151 | RWRKFFKQFF | 33 |
| 166 | FFEHFWKEFN | 34 |
| 167 | FFQHFWKQFN | 35 |
| 168 | QFNHFFKEFF | 36 |
| 169 | FFDKFFHDFQ | 37 |
| 170 | QFDHFFKDFF | 38 |
| 171 | FFEKFFHNFQ | 39 |
| 172 | NFEKWFHEFF | 40 |
| 175 | LFRRAFKQLD | 41 |
| 177 | NFQKWFHQFF | 42 |
| 182 | KFRKAFKRFF | 43 |
| 183 | FFRKFAKRFK | 44 |
| 185 | FFKKFFKKFK | 45 |
| 186 | KFKKFFKKFF | 46 |
| 424 | KARKAFKRFF | 47 |
| 190 | WVKDAMQHLD | 48 |
| 194 | FFKKFAKKFK | 49 |
| 198 | FAEKFFKNFK | 50 |
| 199 | KFNKFFKEAF | 51 |
| 200 | FAKQFFNKFK | 52 |
| 201 | KFNKAFKQAF | 53 |
| 202 | KFNKAFKQAF | 54 |
| 204 | FAQKFFKDFK | 55 |
| 206 | FAEEFAEEFE | 56 |
| 207 | KFKKFFKKAF | 57 |
| 209 | KFKNFFQKAF | 58 |
| 210 | KFKNFFQKAF | 59 |
| 212 | FAKQFANKFK | 60 |
| 213 | KFKNAFQKAF | 61 |
| 214 | KFKNAFQKAF | 62 |
| 215 | FAKKFFKKFK | 63 |
| 216 | KFKKAFKKFF | 64 |
| 218 | FAEKFAEKFE | 65 |
| 219 | DLHQMADKVW | 66 |
| 425 | KARKAAKRFF | 67 |
| 225 | FAKNFAKKFK | 68 |
| 227 | FAEKFAKNFK | 69 |
| 233 | KFKKAFKKAF | 70 |
| 234 | FAKNFAKNFK | 71 |
| 235 | FAKEFAKEFE | 72 |
| 236 | KFDKAFKQAF | 73 |
| 237 | KFDKAFKQAF | 74 |
| 238 | FAEKFAKKFK | 75 |
| 239 | FAEKFAEKFK | 76 |
| 398 | FAKKFAKKFK | 77 |
| 241 | FAKNFAKNFN | 78 |
| 242 | FAQKFAKNFK | 79 |
| 243 | FANNFANNFN | 80 |
| 244 | FANNFANNFN | 81 |
| 245 | FANKFANKFN | 82 |
| 246 | FANKFAKKFK | 83 |
| 247 | FAQKFAKDFK | 84 |
| 250 | FAKEFAKEFK | 85 |
| 251 | FANKFANKFK | 86 |
| 252 | KFDKFFKQAF | 87 |
| 253 | KFDKFFKQAF | 88 |
| 254 | KFNKAFKEAF | 89 |
| 255 | KFNKAFKEAF | 90 |
| 256 | FAKEFAKKFK | 91 |
| 426 | KARKAAKRAF | 92 |
| 427 | KARKAAKRAA | 93 |
| 285 | AAEEAAEEAE | 94 |
| 387 | AAKKAAKKAK | 95 |

In some instances, the CD206 binding peptide is selected from RP124, RP132, RP134, RP142, RP147, RP151, RP166-RP172, RP175, RP177, RP182, RP183, RP185, RP186, RP 424, RP190, RP194, RP198, RP199-RP202, RP204, RP206, RP207, RP209, RP210, RP212-RP216, RP218, RP219, RP425, RP225, RP227, RP233-RP239, RP398, RP241-RP247, RP250-RP256, RP426, RP427, RP285, and RP387. In certain embodiments, the CD206 binding peptide comprises, consists essentially of, or consists of a region of alternating hydrophilic and hydrophobic modules comprising, consisting essentially of, or consisting of a sequence selected from the group consisting of RP124, RP166, RP182, RP183 and RP185.

In some instances, the CD206 binding peptide comprises: a) a peptide sequence selected from RWKFGGFKWR (RP832C) (SEQ ID NO: 96); FWKRFVRKWR (RP837) (SEQ ID NO: 97); FKWRGGRWKF (RP837C) (SEQ ID NO: 98); FWKRGGRKWF (RP837A) (SEQ ID NO: 99); FWKRFV (RP837N) (SEQ ID NO: 100); FWKKFVKKWK (RP841) (SEQ ID NO:101); GDRGIKGHRGF (RP842) (SEQ ID NO: 102); EKLSAFRNFF (RP843) (SEQ ID NO: 103); FYPDFFKKFF (RP844) (SEQ ID NO: 104); FFRHFATHLD (RP845) (SEQ ID NO: 105); LYKKIIKKLL (RP846) (SEQ ID NO: 106); WWHHWWHHWH (RP847) (SEQ ID NO: 107); WWRHWWHRWR (RP848) (SEQ ID NO: 108); WWKHWWHKWK (RP849) (SEQ ID NO: 109); FVRKWR (RP837C1) (SEQ ID NO: 110); FFRKSKEKIG (RP853) (SEQ ID NO: 111); FAOOFAOOFO (RP850) (SEQ ID NO: 112); or b) a sequence having one or two amino acid substitutions relative to the sequence defined in a).

In certain embodiments, the CD206 binding peptide has a sequence having at least 75% sequence identity (e.g., at least 80%, at least 85%, at least 90% or at least 95% sequence identity) with a sequence selected from RWKFGGFKWR (RP832C) (SEQ ID NO: 96); FWKRFVRKWR (RP837) (SEQ ID NO: 97); FKWRGGRWKF (RP837C) (SEQ ID NO: 98); FWKRGGRKWF (RP837A) (SEQ ID NO: 99); FWKRFV (RP837N) (SEQ ID NO: 100); FWKKFVKKWK (RP841) (SEQ ID NO:101); GDRGIKGHRGF (RP842) (SEQ ID NO: 102); EKLSAFRNFF (RP843) (SEQ ID NO: 103); FYPDFFKKFF (RP844) (SEQ ID NO: 104); FFRHFATHLD (RP845) (SEQ ID NO: 105); LYKKIIKKLL (RP846) (SEQ ID NO: 106); WWHHWWHHWH (RP847) (SEQ ID NO: 107); WWRHWWHRWR (RP848) (SEQ ID NO: 108); WWKHWWHKWK (RP849) (SEQ ID NO: 109); FVRKWR (RP837C1) (SEQ ID NO: 110); FFRKSKEKIG (RP853) (SEQ ID NO: 111); FAOOFAOOFO (RP850) (SEQ ID NO: 112). In certain embodiments, the CD206 binding peptide has a sequence having one or two amino acid substitutions relative to a sequence selected from RWKFGGFKWR (RP832C) (SEQ ID NO: 96); FWKRFVRKWR (RP837) (SEQ ID NO: 97); FKWRGGRWKF (RP837C) (SEQ ID NO: 98); FWKRGGRKWF (RP837A) (SEQ ID NO: 99); FWKRFV (RP837N) (SEQ ID NO: 100); FWKKFVKKWK (RP841) (SEQ ID NO:101); GDRGIKGHRGF (RP842) (SEQ ID NO: 102); EKLSAFRNFF (RP843) (SEQ ID NO: 103); FYPDFFKKFF (RP844) (SEQ ID NO: 104); FFRHFATHLD (RP845) (SEQ ID NO: 105); LYKKIIKKLL (RP846) (SEQ ID NO: 106); WWHHWWHHWH (RP847) (SEQ ID NO: 107); WWRHWWHRWR (RP848) (SEQ ID NO: 108); WWKHWWHKWK (RP849) (SEQ ID NO: 109); FVRKWR (RP837C1) (SEQ ID NO: 110); FFRKSKEKIG (RP853) (SEQ ID NO: 111); FAOOFAOOFO (RP850) (SEQ ID NO: 112), wherein the one or two amino acid substitutions are substitutions for amino acids, such as a similar amino acid substitution, a conservative amino acid substitutions or a highly conservative amino acid substitution.

In certain cases, the sequence set forth in a) is RP 832C. In certain cases, the sequence set forth in a) is RP 837. In certain cases, the sequence set forth in a) is RP 837C. In certain cases, the sequence set forth in a) is RP 837A. In certain cases, the sequence set forth in a) is RP 837N. In certain cases, the sequence set forth in a) is RP 837C1. In certain cases, the sequence set forth in a) is RP 841. In certain cases, the sequence set forth in a) is RP 842. In certain cases, the sequence set forth in a) is RP 843. In certain cases, the sequence set forth in a) is RP 844. In certain cases, the sequence set forth in a) is RP 845. In certain cases, the sequence set forth in a) is RP 846. In certain cases, the sequence set forth in a) is RP 847. In certain cases, the sequence set forth in a) is RP 848. In certain cases, the sequence set forth in a) is RP 849. In certain cases, the sequence set forth in a) is RP 850. In certain cases, the sequence set forth in a) is RP 851. In certain cases, the sequence set forth in a) is RP 852. In certain cases, the sequence set forth in a) is RP 853. In some instances, the peptide has the sequence RWKFGGFKWR (RP832C) (SEQ ID NO: 96).

CD206 binding peptides may also be peptides that share a minimum degree of homology with any of the exemplary RP peptides disclosed herein, or variant thereof, or a fragment thereof. Thus, a peptide or polypeptide of the present disclosure is an immunomodulatory peptide that satisfies one of the formulae described herein or shares a minimum degree of homology with any of the exemplary RP peptides disclosed herein. A “fragment” of the invention includes at least 4, 5, 6, 7, 8, 9, 10, 11, 12, 13, 14, 15, 16, 17, 18, 19, 20, 21, 22, or 23 contiguous amino acid residues of a peptide disclosed herein (or up to one less than the number of amino acid residues in the subject peptide) and retains at least one immunomodulatory property of the subject peptide. Thus, fragments of the invention include peptides that are missing one, two, three, four, or more amino acids from the N-terminus and/or the C-terminus relative to a parent immunomodulatory peptide disclosed herein. A “variant” of the invention is a polypeptide that is substantially similar to a polypeptide disclosed herein and retains at least one immunomodulatory property of the subject polypeptide. Variants can include deletions (i.e., truncations) of one or more amino acid residues at the N-terminus or the C-terminus of a subject polypeptide disclosed herein; deletion and/or addition of one or more amino acid residues at one or more internal sites in the subject polypeptide disclosed herein; and/or substitution of one or more amino acid residues (e.g., one, two, three, or even more) at one or more positions in the subject polypeptide disclosed herein. For subject polypeptides that are 12 amino acid residues in length or shorter, variant polypeptides can include three or fewer (e.g., three, two, one, or none) deleted amino acid residues, whether located internally, at the N-terminal end, and/or at the C-terminal end.

CD206 binding peptides that may be employed in embodiments of the invention include peptides that are at least 50% identical (i.e., at least 50% sequence identity) (e.g., at least 60%, at least 70%, at least 80%, at least 85%, at least 90%, at least 95% or more) to any one of the immunomodulatory polypeptides disclosed above and still retain CD206 binding activity. Sequence identity is based on a comparison of two peptide sequences or fragments thereof of the same or similar length. As such, in certain embodiments, CD206 binding peptides are peptides that include an amino acid sequence having from 1 to 10 amino acid differences (e.g., 10 or fewer, 9 or fewer, 8 or fewer, 7 or fewer, 6 or fewer, 5 or fewer, 4 or fewer, 3 or fewer, 2 or fewer, or 1 amino acid difference) to any one of the polypeptides disclosed herein and still retain at least one immunomodulatory property. An “amino acid difference” as used herein includes: an amino acid substitution, an amino acid insertion, a terminal amino acid addition, an amino acid deletion, a terminal amino acid truncation, or any combination thereof. The differences between the a region of alternating hydrophilic and hydrophobic modules of a homologous binding polypeptide and any one of the immunomodulatory polypeptides of Table 3 can include deletions, additions, and/or substitutions of amino acid residues, as discussed herein. Substituted amino acid residues can be unrelated to the amino acid residue being replaced (e.g., unrelated in terms or hydrophobicity/hydrophilicity, size, charge, polarity, etc.), or the substituted amino acid residues can constitute similar, conservative, or highly conservative amino acid substitutions. The determination of whether an amino acid residue substitution is similar, conservative, or highly conservative is based exclusively on the side chain of the amino acid residue and not the peptide backbone, which may be modified to increase peptide stability, as discussed below.

### Label

As reviewed above, in some instances the conjugate includes a label. The label may vary, and in some instances the label is selected from the group consisting of a radionuclide, a radiological contrast agent, a paramagnetic ion, a metal, a biological tag, a fluorescent label, a chemiluminescent label, an ultrasound contrast agent and a photoactive agent. In some instances, label is a radionuclide. Radionuclides of interest include, but are not limited to: ¹¹⁰In, ¹¹¹In, ¹⁷⁷Lu, ¹⁸F, ⁵²Fe, ⁶²Cu, ⁶⁴Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

Where desired, the radionuclide may be complexed with a chelator, where the chelator is bonded to the CD206 binding peptide, e.g., as illustrated in FIG. 1. Any convenient chelator may be employed. Chelators of that may be employed include, but are not limited to, DTPA, DO3A, DOTA, EDTA, TETA, EHPG, HBED, NOTA, DOTMA, TETMA, PDTA, TTHA, LICAM, HYNIC, and MECAM. In some instances, the chelator is DOTA, which has the structure:

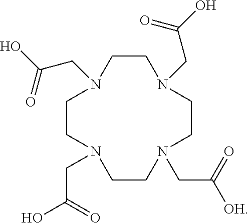 [SMILES: O=C(O)CN1CCN(CC(=O)O)CCN(CC(=O)O)CCN(CC(=O)O)CC1]

Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. Pat. No. 7,563,433, the disclosure of which is incorporated by reference). Useful chelators encompassed by the invention include, but are not limited to, DTPA, DO3A, DOTA, EDTA, TETA, EHPG, HBED, NOTA, DOTMA, TETMA, PDTA, TTHA, LICAM, HYNIC, and MECAM.

Labels of interest may also include non-radionuclide labels. In some instances, the label is a metal. A number of metals useful for MRI include gadolinium, manganese, copper, iron, gold and europium. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). CD206 binding peptides can also be made detectable by coupling them to a phosphorescent or a chemiluminescent compound. The presence of the chemiluminescent-tagged peptide is then determined by detecting the presence of luminescence that arises during the course of a chemical reaction. Examples of particularly useful chemiluminescers are luminol, isoluminol, theromatic acridinium ester, imidazole, acridinium salt and oxalate ester. Likewise, a bioluminescent compound may be used to label the peptides. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. In yet another embodiment, colorimetric detection is used, based on chromogenic compounds which have, or result in, chromophores with high extinction coefficients.

In situ detection of the labeled peptide may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

### Therapeutic Agent

As reviewed above, in some instances the conjugate includes a therapeutic agent. Therapeutic agents of interest include, but are not limited to cytotoxic agents, anti-angiogenic agents, pro-apoptotic agents, antibiotics, hormones, hormone antagonists, chemokines, drugs, prodrugs, toxins, enzymes, antimitotics, antikinases, alkylating agents, antimetabolites and alkaloids.

Of interest in certain embodiments are cytotoxic radionuclides. Cytotoxic radionuclides that are present in conjugates of embodiments of the invention may be radionuclides are those that emit Auger electrons, radionuclides emit either beta-(β⁻)-or alpha (α)-particles, etc. Cytotoxic radionuclides of interest include, but are not limited to: ²²⁵Ac, ²¹¹At, ²¹³Bi, ²¹²Pb, ¹³¹I, ¹⁷⁷Lu, ¹⁸⁸Re, ⁹⁰Y, ¹¹¹In, ¹²⁵I.

Where desired, the therapeutic radionuclide may be complexed with a chelator, where the chelator is bonded to the CD206 binding peptide, e.g., as illustrated in FIG. 1. Any convenient chelator may be employed. Chelators of that may be employed include, but are not limited to, DTPA, DO3A, DOTA, EDTA, TETA, EHPG, HBED, NOTA, DOTMA, TETMA, PDTA, TTHA, LICAM, HYNIC, and MECAM. In some instances, the chelator is DOTA, which has the structure:

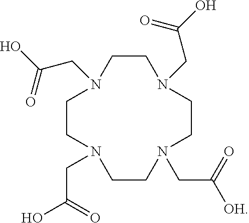 [SMILES: O=C(O)CN1CCN(CC(=O)O)CCN(CC(=O)O)CCN(CC(=O)O)CC1]

Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. Pat. No. 7,563,433, the disclosure of which is incorporated by reference). Useful chelators encompassed by the invention include, but are not limited to, DTPA, DO3A, DOTA, EDTA, TETA, EHPG, HBED, NOTA, DOTMA, TETMA, PDTA, TTHA, LICAM, HYNIC, and MECAM.

In some instances, the therapeutic agent is a cancer chemotherapeutic agent. Cancer chemotherapeutic agents include non-peptidic (i.e., non-proteinaceous) compounds that reduce proliferation of cancer cells and encompass cytotoxic agents and cytostatic agents. In certain cases, the chemotherapeutic agent, antibody agent or cell therapy is selected from steroids, anthracyclines, thyroid hormone replacement drugs, thymidylate-targeted drugs, antibodies, checkpoint inhibitor drugs, Chimeric Antigen Receptor/T cell therapies, and other cell therapies. Non-limiting examples of chemotherapeutic agents include alkylating agents, nitrosoureas, antimetabolites, antitumor antibiotics, plant (vinca) alkaloids, and steroid hormones. Peptidic compounds can also be used. Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD). Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide. Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine. Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like. Other anti-proliferative cytotoxic agents are navelbene, CPT-11, anastrazole, letrazole, capecitabine, reloxafine, cyclophosphamide, ifosamide, and droloxafine. Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, epothilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation. Therefore, compounds that bind to the estrogen receptor are used to block this activity. Corticosteroids may inhibit T cell proliferation. Other suitable chemotherapeutic agents include metal complexes, e.g., cisplatin (cis-DDP), carboplatin, etc.; ureas, e.g., hydroxyurea; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose). Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701. Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

In some instances, the therapeutic agent comprises an antibody. Suitable antibodies for use in cancer treatment include, but are not limited to, naked antibodies, e.g., trastuzumab (Herceptin), bevacizumab (Avastin™), cetuximab (Erbitux™), panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), 90Y-labelled ibritumomab tiuxetan (Zevalin™), 131I-labelled tositumoma (Bexxar™), etc. Suitable antibodies for use in cancer treatment include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, etc.

In some instances, the therapeutic agent comprises an immune checkpoint inhibitor, like anti-CTLA4 or anti-PD-1 and anti-PD-1L agents. The immune system depends on multiple checkpoints to avoid over-activation of the immune system on healthy cells, and tumor cells often take advantage of these checkpoints in order to escape detection by the immune system. CTLA-4, shown to be aberrantly upregulated and present on the surface of T cells in certain cancers, and PD-1, also upregulated in certain tumors and found to inhibit T-cell function, are checkpoints that have been studied as targets for cancer therapy (Pardoll, D. M. 2012 Nat Rev Cancer 12(4):252-264; Sharma, et al. 2011 Nat Rev Cancer 11(11):805-812).

Additional details regarding therapeutic agents that may be conjugated to CD206 binding peptides in accordance with embodiments of the invention may be found in U.S. patent application Ser. No. 17/478,042, the disclosure of which is herein incorporated by reference.

### Compositions

In embodiments, compositions for use in diagnosing or treating a subject according to the present disclosure, e.g., as described below, can be formulated according to any of the conventional methods known in the art and widely described in the literature. Thus, a CD206-targeted peptide conjugate, e.g., as described herein, may be incorporated, optionally together with other active substances, with one or more conventional pharmaceutically acceptable carriers, diluents and/or excipients, etc., appropriate for the particular use of the composition, to produce conventional preparations that are suitable or can be made suitable for administration. Such compositions may be formulated as liquids, as semi-solids or solids, liquid solutions, dispersions, suspensions, and the like, depending on the intended mode of administration and therapeutic application. In some embodiments, the composition is prepared in a form of an injectable or infusible solution.

In certain embodiments of the inventive treatment methods, administration is via any one of a variety of routes, including intravenous (IV), intramuscular (IM), intra-arterial, intramedullary, intrathecal, subcutaneous (SQ), intraventricular, transdermal, interdermal, intradermal, intratumoral, by intratracheal instillation, bronchial instillation, and/or inhalation; as a nasal spray, and/or aerosol, and/or through a portal vein catheter. In certain embodiments, intravenous injection, or infusion may be used. Any appropriate site of administration may be used. For example, the inventive composition may be administered locally and directly at the site where action is required or may be attached or otherwise associated, e.g., conjugated, with entities which will facilitate the targeting to an appropriate location in the body.

In certain embodiments, any physiologically compatible carrier, excipient, diluent, buffer or stabilizer can be used in the compositions of the invention. Examples of suitable carriers, excipients, diluents, buffers and stabilizers include one or more of water, saline, phosphate buffered saline, dextrose, glycerol, ethanol and the like, as well as combinations thereof. In some cases isotonic agents, e.g., sugars, polyalcohols (e.g., mannitol, sorbitol), or sodium chloride may be included. In certain embodiments, the compositions of the invention may be formulated so as to provide quick, sustained, or delayed release of the active ingredient (peptide A, peptide B, or variants thereof and/or additional drug(s)) after administration to the subject by employing procedures well known in the art. As described above, in certain embodiments, the composition is in a form suitable for injection and suitable carriers may be present at any appropriate concentration, but exemplary concentrations are from 1% to 20% or from 5% to 10%.

Compositions of the invention may be sterile and stable under conditions of manufacture and storage. Appropriate ways of achieving such sterility and stability are well known and described in the art.

Compositions of the invention may be formulated in unit dosage form for ease of administration and uniformity of dosage. It will be understood, however, that the total daily (or other) usage of the compositions of the present invention will be decided by the attending physician within the scope of sound medical judgment. The specific therapeutically effective dosage level for any particular subject will depend upon a variety of factors including the activity of the composition employed; the half-life of the composition after administration; the age, body weight, general health, sex, and diet of the subject; the time of administration, route of administration, and rate of excretion of peptide A and (if used) the additional therapeutic agent employed; the duration of the treatment; drugs used in combination or coincidental with the specific compound employed; and like factors, well known in the medical arts. Furthermore, effective doses may be extrapolated from dose-response curves derived from in vitro and/or in vivo animal models.

Thus, suitable doses of the CD206 targeted peptide conjugates and other active ingredients (if included) will vary from patient to patient. In some embodiments, said dosages constitute a therapeutically effective amount or a prophylactically effective amount, depending on the nature of the treatment involved. The ability of the CD206 targeted peptide conjugate to elicit a desired response in the individual will also be a factor. Exemplary daily doses are: 0.1 to 250 mg/kg, or 0.1 to 200 or 100 mg/kg, or 0.5 to 100 mg/kg, or 1 to 50 or 1 to 10 mg/kg, of the active ingredient. This can be administered as a single unit dose or as multiple unit doses administered more than once a day, for example, subcutaneously, intraperitoneally, or intravenously. It is to be noted, however, that appropriate dosages may vary depending on the patient, and that for any particular subject, specific dosage regimes should be adjusted over time according to the individual needs of the patient. Thus, the dosage ranges set forth herein are to be regarded as exemplary and are not intended to limit the scope or practice of the claimed compositions or methods.

### Methods

Aspects of the invention further include methods of using the conjugates. CD206 targeted peptide conjugates find use in a variety of different applications, where such applications include, but are not limited to, diagnostic and therapeutic applications.

### Diagnostic Applications

As summarized above, CD206 targeted peptide conjugates find use in diagnostic applications. In embodiments of diagnostic applications, the CD206 targeted peptide conjugates are conjugated to a label, e.g., as described above, where such conjugates may be administered to a subject and then imaged, e.g., for diagnostic applications.

In one aspect, the invention provides in vivo methods and compositions for diagnosing a disease condition, e.g., cancer, fibrosis, etc. The methods include identifying a subject at risk for or suspected of having the disease condition, e.g., cancer or fibrosis; administering to a subject a diagnostic composition comprising a CD206 targeted peptide conjugate of the invention conjugated to or comprising one or more imaging molecules, and imaging the imaging molecule within the subject using in vivo imaging. In some embodiments, the composition is administered via route selected from the group consisting of intradermal, subcutaneous, intraperitoneal, intravenous, intraarterial, oral, and gastric routes. In some embodiments, the in vivo imaging includes but is not limited to magnetic resonance imaging (MRI), intravital laser scanning microscopy, endoscopy, PET, SPECT/CT, and radiographic imaging. Imaging techniques of interest include, but are not limited to, fluorescence, positron emission tomography (PET), magnetic resonance imaging (MRI), single photon emission computed tomography (SPECT/CT), intravital laser scanning microscopy, endoscopy, and radiographic imaging.

The invention further provides for monitoring the progression of a disease condition, e.g., cancer or fibrosis. In one embodiment, the present invention further provides compositions and methods for monitoring the progression or treatment of a disease condition, e.g., cancer or fibrosis. With respect to cancer, embodiments include methods of determining whether patients are responsive to immune checkpoint therapy. Embodiments include methods of determining whether patients are responsive Receptor Tyrosine Kinase/MAPK, ERK (MEK) inhibitors, PARP inhibitors therapy. Embodiments include methods of predicting metastatic cancer. With respect to fibrosis, embodiments of the invention include methods of diagnosing whether scleroderma disease is progressing to the lungs. Embodiments include methods of predicting whether a patient may suffer from, or diagnosing whether a patient suffers from, Rapidly Progressive Interstitial Lung Disease.

In some instances, CD206 targeted peptide conjugates of the invention are conjugated to a radionuclide label, e.g., as described above. Following administration of such conjugates to a subject, an imaging protocol such as positron electron tomography (PET) is employed to image the administered conjugates. The resultant image data may then be employed in diagnosis of a disease condition, e.g., cancer, fibrosis, etc.

Positron emission tomography is a nuclear medicine imaging technique which produces a three-dimensional image or picture of functional processes in the body. The theory behind PET is as follows. First a molecule is tagged with a positron-emitting isotope. These positrons annihilate with nearby electrons, emitting two 511 keV photons, directed 180 degrees apart in opposite directions. These photons are then detected by the scanner which can estimate the density of positron annihilations in a specific area. When enough interactions and annihilations have occurred, the density of the original molecule may be measured in that area. Typical isotopes include ¹¹C, ¹³N, ¹⁵O, ¹⁸F, ⁶⁴Cu, ⁶²Cu, ¹²⁴I, ⁷⁶Br, ⁸²Rb and ⁶⁸Ga, with ¹⁸F being the most clinically utilized. Although some PET probes must be made with a cyclotron and have a half-life measured in hours, forcing the cyclotron to be on site, PET imaging does have many advantages though. First and foremost is its sensitivity: a typical PET scanner can detect between 10⁻¹¹ mol/L to 10⁻¹² mol/L concentrations. Some agents used for PET imaging provide information about tissue metabolism or some other specific molecular activity. Following are commonly used agents or potential agents for use with the compositions and methods of the invention or in combination with the other compositions and methods of the invention:
⁶⁴Cu-ATSM: ⁶⁴Cu diacetyl.bis(N⁴-methylthiosemicarbazone), also called ATSM or Cooper 64, is an imaging agent used in PET or PET/CT for its ability to identify hypoxic tissue (tissue with low oxygen).
FDG: ¹⁸F-fluorodeoxyglucose (FDG) is a radioactive sugar molecule, that, when used with PET imaging, produces images that show the metabolic activity of tissues. In FDG-PET scanning, the high consumption of the sugar by tumor cells, as compared to the lower consumption by normal surrounding tissues, identifies these cells as cancer cells. FDG is also used to study tumor response to treatment.
¹⁸F-fluoride: ¹⁸F-fluoride is an imaging agent for PET imaging of new bone formation. It can assess changes both in normal bone as well as bone tumors. As a result, it can be used to measure response to treatment.
FLT: 3′-deoxy-3′-[¹⁸F]fluorothymidine (FLT) is a radiolabeled imaging agent that is being investigated in PET imaging for its ability to detect growth in a primary tumor. Studies may also measure the ability of FLT with PET to detect tumor response to treatment.
FMISO: ¹⁸F-fluoromisonidazole is an imaging agent used with PET imaging that can identify hypoxia (low oxygen) in tissues. Tumors with low oxygen have been shown to be resistant to radiation and chemotherapy.
Gallium: Gallium attaches to areas of inflammation, such as infection. It also attaches to areas of rapid cell division, such as cancer cells. It can take gallium a few days to accumulate in the affected tissue, so the scan may be done 2-3 days after the gallium is administered.
Technetium-99m: Technetium-99m is used to radiolabel many different common radiopharmaceuticals. It is used most often in bone and heart scans.
Thallium: Thallium is a radioactive tracer typically used to examine heart blood flow. The thallium scan is often combined with an exercise test to determine how well the heart functions under stress. A thallium scan may also be used to measure tumor response.

Radiopaque diagnostic agents may be selected from compounds, barium compounds, gallium compounds, and thallium compounds.

A wide variety of fluorescent labels are known in the art, including but not limited to fluorescein isothiocyanate, rhodamine, phycoerythrin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

Techniques for detecting and measuring these agents are provided in the art or described herein. Detecting the location of the imaging agent may be conducted by any suitable technique known to one skilled in the art, for example, by positron emission tomography (PET).

Yet another modification may comprise the introduction of one or more detectable labels or other signal-generating groups or moieties, depending on the intended use of the labeled molecule. Suitable labels and techniques for attaching, using and detecting them will be understood by one of ordinary skill in the art, and for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isolurinol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malale dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be understood by the skilled artisan, and for example, include moleties that can be detected using NMR or ESR spectroscopy. Such labeled molecules of the invention may, for example, be used for in vitro, in vivo or in situ assays (including immunoassays known per se such as ELISA, RIA, ElA and other “sandwich assays,” etc.) as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA), Yet another modification may comprise the introduction of a functional group that is one part of a specific binding pair, such as the biotin-(strept)avidin binding pair. Such a functional group may be used to link a molecule of the invention to a protein, polypeptide or chemical compound that is bound to the other half of the binding pair, i.e., through formation of the binding pair. For example, such a conjugated molecule may be used as a reporter, for example, in a diagnostic system where a detectable signal-producing agent is conjugated to avidin or streptavidin.

A number of trivalent metal radionuclides have physical properties suitable for radioisotope imaging (e.g., indium-111 (¹¹¹In) gallium-67/68 (^{67/68}Ga) and yttrium-86 (⁸⁶Y)) or for targeted radionuclide therapy (e.g., ⁹⁰Y and lutetium-177 (¹⁷⁷Lu). These metal radionuclides can be employed in conjugates of the invention in order to diagnose, monitor or treat disease.

Additional details of types of labels or therapeutic agents and diagnostic methods of using the same may be found in United States Published Patent Application 20130330274; the disclosure of which is herein incorporated by reference.

### Therapeutic Applications

Embodiments of the invention provide methods of a treating a subject for a disease condition or disorder. Disease conditions or disorders may vary. In some instances the disease condition is cancer. In some instances, the disease condition is fibrosis.

### Cancer

Embodiments of the Invention provide a method of treating a subject with cancer, comprising, a) providing: i) a subject in need of treatment; ii) a pharmaceutical composition comprising a CD206 targeted peptide conjugate of the invention; and b) administering the treatment composition to the subject. In some embodiments, the pharmaceutical composition further comprises a therapeutic agent, e.g., as described above.

Various types of cancer may be treated using CD206 targeted peptide conjugates in accordance with embodiments of the invention. Cancers of interest for treatment according to embodiments of the present disclosure include but are not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy.

With respect to the cancer as a whole, desired effects of the described treatments may result in a reduction in the number of cells in the cancer, a reduction in the size of a tumor, a reduction in the overall proliferation of the cancer, a reduction in the overall growth rate of a tumor, etc. For example, an effective treatment is in some cases a treatment that, when administered in one or more doses to an individual in need thereof, reduces the number of cancer cells in the individual and/or reduces tumor mass in the individual, by at least about 10%, at least about 15%, at least about 20%, at least about 25%, at least about 30%, at least about 40%, at least about 50%, at least about 75%, or more than 75%, compared to the number of cancer cells and/or tumor mass in the absence of the treatment.

In some embodiments, an effective treatment is a treatment that, when administered alone (e.g., in monotherapy) or in combination (e.g., in combination therapy) with one or more additional therapeutic agents, in one or more doses, is effective to reduce one or more of tumor growth rate, cancer cell number, and tumor mass, by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the tumor growth rate, cancer cell number, or tumor mass in the absence of the treatment.

In another embodiment, the present invention provides methods for surgically removing a tumor(s). The methods include a) providing: i) a composition comprising a CD206 targeted peptide conjugate of the invention for distinguishing a cancer ii) a subject known to have cancer; ill) an in vivo imaging device; and b) administering the composition to a subject; c) imaging TAMS to identify the cancer in vivo with the imaging device; and d) removing the tumor from the subject following detecting their location.

Additional details of types of labels or therapeutic agents and therapeutic methods of using the same may be found in United States Published Patent Application 20130330274; the disclosure of which is herein incorporated by reference.

### Fibrosis

Embodiments of the invention include methods for preventing and/or treating fibrosis. Fibrosis is an inflammatory disease in which inflammatory cells migrate into tissue and organs, leading to cellular responses that result in scarring. Fibrosis can occur in many tissues within the body, typically as a result of inflammation or damage. By preventing inflammatory cell extravasation, fibrosis can be attenuated or prevented.

M2 polarized macrophages (to which CD206 targeted peptide conjugates of the invention bind) secrete large amounts of transforming growth factor beta, or TGFβ, an important cytokine involved in cellular signaling, which induces fibroblast accumulation leading to collagen deposition and tissue remodeling, ultimately resulting in tissue fibrosis. Reprogramming these M2 macrophages to reduce TGFβ production by targeting key signaling molecules, like STAT3, has been shown to have beneficial activity in preclinical models of lung fibrosis.

Also provided herein are methods of treating a fibrotic condition in a subject comprising administering to the subject in need thereof an effective amount of a composition comprising a CD206 targeting peptide conjugate. In some embodiments, the fibrotic condition is lung fibrosis, liver fibrosis, or pancreatic fibrosis. In certain embodiments, the liver fibrosis is non-alcoholic steatohepatitis, or NASH. See, e.g., See Mayo Clinic Staff. “Definition [of pulmonary fibrosis]”. Mayo Foundation for Medical Education and Research. Archived from the original on 15 Jul. 2014. Retrieved 26 Jul. 2014, available from the world wide at webmayoclinic.org/diseases-conditions/pulmonary-fibrosis/symptoms-causes/syc-20353690; Ferri F. Idiopathic pulmonary fibrosis. In: Ferri's Clinical Advisor 2016. Philadelphia, Pa.: Mosby Elsevier; 2016; Gross T J, Hunninghake G W (2001). “Idiopathic pulmonary fibrosis”. N Engl J Med. 345 (7): 517-525; Friedman L S (2014). Current medical diagnosis and treatment 2014. [S.1]: Mcgraw-Hill, pp. Chapter 16. Liver, Biliary Tract, & Pancreas Disorders; Chalasani N, Younossi Z, Lavine J E, Charlton M, Cusi K, Rinella M, Harrison S A, Brunt E M, Sanyal A J (January 2018). “The diagnosis and management of nonalcoholic fatty liver disease: Practice guidance from the American Association for the Study of Liver Diseases”. Hepatology. 67 (1): 328-357; Xue J, Sharma V, Hsieh M H, Chawla A, Murali R, Pandol S J, Habtezion A. Nat Commun. 2015 May 18; 6:7158.

In one embodiment, the CD206 targeted peptide conjugates and methods provided herein can be used to prevent and/or treat pulmonary fibrosis. In lungs, types of fibrosis include pulmonary fibrosis such as cystic fibrosis and idiopathic pulmonary fibrosis. Pulmonary fibrosis is a respiratory disease in which scars are formed in the lung tissues, leading to serious breathing problems. Scar formation leads to thickening of the walls, and causes reduced oxygen supply in the blood. As a consequence, patients suffer from perpetual shortness of breath.

In one embodiment, the CD206 targeted peptide conjugates and methods provided herein can be used to treat liver fibrosis. Liver fibrosis may result from a wide variety of conditions including chronic alcohol exposure, hepatitis B virus (HBV) infection, non-alcoholic fatty liver disease (NAFLD), Nonalcoholic steatohepatitis (NASH), hepatitis C virus (HCV) infection, Wilson's disease, alpha-1-antitrypsin deficiency, hemochromatosis, primary biliary cirrhosis, primary sclerosing cholangitis, and autoimmune hepatitis. Chronic HCV is the leading contributor to chronic liver disease and the liver elicits a persistent inflammatory and fibrosis, which is characterized by the formation of fibrous tissue and scarring on the liver. NAFLD and NASH also cause inflammation and fibrosis in the liver.

Cirrhosis is fibrosis in the liver in which the liver does not function properly due to long-term damage. Typically, the disease comes on slowly over months or years. Early on, there are often no symptoms. As the disease worsens, a person may become tired, weak, itchy, have swelling in the lower legs, develop yellow skin, bruise easily, have fluid buildup in the abdomen, or develop spider-like blood vessels on the skin. The fluid build-up in the abdomen may become spontaneously infected. Other complications include hepatic encephalopathy, bleeding from dilated veins in the esophagus or dilated stomach veins, and liver cancer. Hepatic encephalopathy results in confusion and possibly unconsciousness. Cirrhosis can result in liver dysfunction. The following symptoms or features are direct consequences of liver dysfunction and thus can also be treated or ameliorated by the presently disclosed compositions and methods.

In one embodiment, the CD206 targeted peptide conjugates and methods provided herein can be used to prevent and/or treat renal fibrosis. Renal fibrosis can result from acute or sustained injury to the kidney. The injury can lead to excessive deposition of extracellular matrix. Over time, this can result in kidney failure, requiring patients to undergo dialysis or kidney transplant.

In one embodiment, the CD206 targeted peptide conjugates and methods provided herein can be used to prevent and/or treat fibrosis in the heart. Fibrosis in the heart is present in the form of atrial fibrosis, endomyocardial fibrosis, or myocardial infarction. Glial scar is fibrosis in the brain. Other types of fibrosis include, without limitation, arthrofibrosis (knee, shoulder, other joints), Crohn's disease (intestine), Dupuytren's contracture (hands, fingers), keloid (skin), mediastinal fibrosis (soft tissue of the mediastinum), myelofibrosis (bone marrow), Peyronie's disease (penis), nephrogenic systemic fibrosis (skin), progressive massive fibrosis (lungs), retroperitoneal fibrosis (soft tissue of the retroperitoneum), scleroderma/systemic sclerosis (skin, lungs), and some forms of adhesive capsulitis (shoulder).

CD206 targeted peptide conjugates and methods of the present disclosure are suitable for preventing and/or treating any of these diseases or symptoms or features associated with these diseases. Accordingly, the CD206 targeted peptide conjugates can prevent, inhibit, delay, and/or reverse fibrosis. In certain embodiments, the fibrosis is post ischemic, post infectious, or idiopathic (e.g., renal, hepatic, cardiac, pulmonary). See, e.g., Guerrot, D., et al. Fibrogenesis & tissue repair 5.Suppl 1 (2012): S15, and Yamaguchi, I, et al. Nephron Experimental Nephrology 120.1 (2012): e20-e31. In certain embodiments, the fibrosis is retroperitoneal. In certain embodiments, the fibrosis is dermal (e.g., scleroderma). See, e.g., Maurer, B., et al. Annals of the rheumatic diseases (2013): annrheumdis-2013.

In one embodiment, provided herein is a use of the CD206 targeted peptide conjugates disclosed herein for the prevention or treatment of fibrosis. In one embodiment, provided herein is a use of the CD206 targeted peptide conjugates disclosed herein for the preparation of a medicament for the prevention or treatment of fibrosis. In one embodiment, provided herein is a use of the CD206 targeted peptide conjugates disclosed herein for the prevention or treatment of liver fibrosis. In one embodiment, provided herein is a use of the CD206 targeted peptide conjugates disclosed herein for the prevention or treatment of pulmonary fibrosis.

Additional details regarding treatment of fibrosis in which the CD206 targeted binding peptide conjugates of the invention may find use in accordance with embodiments of the invention may be found in U.S. patent application Ser. No. 17/478,042, the disclosure of which is herein incorporated by reference.

### Administration

In some embodiments, the composition is administered intravenously to the circulatory system of the subject. In some embodiments, the composition is infused in suitable liquid and administered into a vein of the subject. In some embodiments, the composition is administered intra-arterially to the circulatory system of the subject. In some embodiments, the composition is infused in suitable liquid and administered into an artery of the subject. In some embodiments, the composition is administered to the subject by intrathecal administration. In some embodiments, the composition is administered via an injection into the spinal canal, or into the subarachnoid space so that it reaches the cerebrospinal fluid (CSF). In some embodiments, the composition is administered to the subject by intranasal administration. In some embodiments, the composition can be insufflated through the nose in a form of either topical administration or systemic administration. In certain embodiments, the composition is administered as nasal spray. In some embodiments, the composition is administered to the subject by intraperitoneal administration. In some embodiments, the composition is infused in suitable liquid and injected into the peritoneum of the subject. In some embodiments, said intraperitoneal administration results in distribution of the composition to the lymphatics. In some embodiments, the intraperitoneal administration results in distribution of the composition to the thymus, spleen, and/or bone marrow. In some embodiments, said intraperitoneal administration results in distribution of the composition to one or more lymph nodes. In some embodiments, said intraperitoneal administration results in distribution of the composition to one or more of the cervical lymph node, the inguinal lymph node, the mediastinal lymph node, or the sternal lymph node. In some embodiments, said intraperitoneal administration results in distribution of the composition to the pancreas. In some embodiments, the composition is administered to the subject by periocular administration. In some embodiments, the composition is injected into the periocular tissues. Periocular drug administration includes the routes of subconjunctival, anterior sub-Tenon's, posterior sub-Tenon's, and retrobulbar administration. In some embodiments, the composition is administered into the same subject by multiple routes of administration. In some embodiments, said multiple routes of administration comprise intravenous administration, intra-arterial administration, intrathecal administration, intranasal administration, intraperitoneal administration, and/or periocular administration. In a preferred embodiment, said multiple routes of administration comprise intravenous administration and intraperitoneal administration.

In certain embodiments, the dosage of the extracellular vesicles is between 1 ng to 10 ng, 10 ng to 100 ng, 100 ng to 1 μg, 1 μg to 5 μg, 5 μg to 10 μg, 10 μg to 50 μg, 50 μg to 75 μg, 75 μg to 100 μg, 100 μg to 150 μg, 150 μg to 200 μg, 200 μg to 300 μg, 300 μg to 500 μg, 500 μg to 1 mg, or 1 mg to 10 mg.

The compositions can be administered once to the subject. Alternatively, multiple administrations can be performed over a period of time. For example, two, three, four, five, or more administrations can be given to the subject. In some embodiments, administrations can be given as needed, e.g., for as long as symptoms associated with the disease, disorder or condition persists. In some embodiments, repeated administrations can be indicated for the remainder of the subject's life. Treatment periods can vary and can be, e.g., no longer than a year, six months, three months, two months, one month, two weeks, one week, three days, two days, or no longer than one day. In certain embodiments, doses are administered at intervals such as once daily, every other day, once weekly, twice weekly, once monthly or twice monthly. In some embodiments, the pharmaceutical composition is administered at a frequency sufficient to effectively increase the concentration of the immunomodulating component in the target cell or tissue above a level that is associated with a symptom of the disease, disorder or condition. In some embodiments, the compositions are administered at least twice over a treatment period such that the disease, disorder or condition is treated, or a symptom thereof is ameliorated. In some embodiments, the compositions are administered at least twice over a treatment period such that the disease, disorder or condition is treated or a symptom thereof is prevented. In some embodiments, the pharmaceutical composition is administered a sufficient number of times over a treatment period such that a sufficient amount of immunomodulating component is delivered to the target cell or tissue during the treatment period. In some embodiments, the pharmaceutical composition is administered a sufficient number of times over a treatment period such that a sufficient amount of immunomodulating component is delivered to the target cell or tissue during the treatment period such that one or more symptoms of the disease, disorder or condition is prevented, decreased, ameliorated or delayed. In some embodiments, increasing the immunomodulating component concentration in the target cell or tissue includes increasing the peak concentration, while in others it includes increasing the average concentration. In some embodiments, a substantial increase during the treatment period can be determined by comparing a pretreatment or post-treatment period in the subject, or by comparing measurements made in a population undergoing treatment with a matched, untreated control population. In some embodiments, the pharmaceutical composition is administered a sufficient number of times per treatment period such that the concentration of immunomodulating component in the target cell or tissue is increased for at least about one week, two weeks, three weeks, four weeks, one month, two months, three months, four months, five months, six months or greater than six months. In some embodiments, the pharmaceutical composition is administered a sufficient number of times per treatment period such that the concentration of immunomodulating component in the target cell or tissue is increased for a period of time at least as long as the treatment period. In some embodiments, the time interval between repeated administrations within a treatment period is no longer than the period in which the number of extracellular vesicles in circulation is reduced to less than about 5%, 10%, 15%, 20%, 25%, 30%, 35%, 40%, 45%, 50%, 55%, 60%, 65%, 70%, 75%, 80%, 85%, 90%, or 95% of the number of extracellular vesicles present in the administered pharmaceutical composition. The dosing and frequency of the administration of the extracellular vesicles and pharmaceutical compositions thereof can be determined, e.g., by the attending physician based on various factors such as the severity of disease, the patient's age, sex and diet, the severity of any inflammation, time of administration and other clinical factors. In an example, an intravenous administration is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that can appear.

### Kits

In one aspect, the present disclosure further provides kits the CD206 targeted peptide conjugate, or a composition formulated with the CD206 targeted peptide conjugate. Kits can include one or more other elements including, but not limited to, instructions for use; other therapeutic agents (for combination therapy); other reagents, e.g., a diluent, devices or other materials for preparing composition for administration; pharmaceutically acceptable carriers; and devices or other materials for administration to a subject. Instructions for use can include instructions for therapeutic application, including suggested dosages and/or modes of administration, e.g., in a human subject, as described herein. In some embodiments, said kits are for use in the methods and uses as described herein, e.g., therapeutic, diagnostic, or imaging methods, or are for use in in vitro assays or methods. The peptide(s) or variant in such kits may, in some embodiments, be a conjugate, e.g., may be conjugated to a detectable moiety.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example I. Evaluation of a CD206 Targeted Peptide for PET Imaging of Macrophages in Syngeneic Mouse Models of Cancer

### A. MATERIALS AND METHODS

### 1. General Methods

All reagents and solvents were purchased from Fisher Scientific and Sigma-Aldrich unless stated otherwise. RP-832c (RWKFGGFKWR) and MART-1(ELAGIGILTV) with and without 1,4,7,10-tetraazacyclododecane-1,4,7,10-tetraacetic acid (DOTA) were synthesized on a commercially solid phase peptide synthesizer with purities greater than 95 percent for each peptide. An AR-2000 Eckert and Ziegler Thin Layer Chromatography (TLC) Scanner and an Agilent Technologies 1260 Infinity High Performance Liquid Chromatography (HPLC) were used to determine radiochemical yields and purities.

### 2. Radiochemistry

Radiolabeling with ⁶⁸Ga (t_{1/2}=68 min; β⁺=89%) was accomplished using a previously published protocol.¹³ The general procedure was as follows: ⁶⁸GaCl₃ (5-10 mCi) was obtained from a commercial ⁶⁸Ge/⁶⁸Ga generator (GalliaPharm, Eckert & Ziegler) by eluting with 10 mL of 0.1 M HCL through a SCX cartridge (Bond Elut SCX Cartridges, 100 mg, particle size 40 μm) and then dried with a stream of air. The flow rate for the elution was set to 1 mL per minute. 200 μL of a 5 M NaCl/HCl solution (7.3 g of NaCl and 625 μL of 5.5 HCL in 25 ml of water) was used for the elution of ⁶⁸GaCl₃ from the SCX cartridge.

Labeling of RP-832 and MART-1 with ⁶⁸Ga was accomplished by adding 10 μg of peptide to 500 μCi of ⁶⁸GaCl₃ in 50 μL of 1 M sodium acetate buffer (pH 4.5). The reactions were incubated in a thermomixer at 95 degrees Celsius for 14 minutes with 300 rpm agitation. Incorporation of the ⁶⁸Ga was determined by radio-High Performance Liquid Chromatography (HPLC) immediately after the reaction. Analysis was performed using a C-18 column. The gradient elution system utilized mobile phase A (100% water 0.1% TFA) and mobile phase C (Acetonitrile containing 0.1% TFA) with a flow rate of 1.0 mL/min. The gradient started with 100% A and was increased to 100% C over 6.67 minutes and then returned to the initial gradient conditions within 4.66 minutes.

### 3. In Vitro Stability

250 μL (˜2.5 mCi) of each ⁶⁸Ga labeled peptide (RP-832c and MART-1; 200 μg) was added to either 400 μL of mouse serum or phosphate-buffered saline (PBS). The mixtures were incubated at 37 degrees Celsius with 400 rpm agitation up to three hours. Aliquots were removed at different time points (30 minutes, 1, 2, 3 h) to evaluate stability and analyzed using radio-HPLC as described above. All experiments were completed in triplicate.

### 4. CD206 Binding Affinity of [⁶⁸Ga]RP832c and [⁶⁸Ga]MART-1

The in vitro binding characteristics of ⁶⁸Ga-RP832c to CD206 were determined by a protein plate binding assay. CD206 mouse protein was purchased from R&D Systems and adhered to a 96 well high-binding single-break strip plate (Fisher Scientific) using a 15 mM sodium carbonate, 35 mM sodium bicarbonate coating buffer (1 μg of protein per well) and refrigerated at 4 degrees Celsius for 48 hours. After 48 hours, 100 μL (50 nM) of [⁶⁸Ga]RP832c and [⁶⁸Ga]MART-1 and 100 μL of PBS, was added to the wells on columns 1 and 3 respectively. In order to obtain CD206 blocking data, 100 μL (5 M) of unlabeled RP-832c and MART-1, along with 100 μL (50 nM) of [⁶⁸Ga]RP832c and [⁶⁸Ga]MART-1, was added to columns 2 and 4, respectively. The plate was allowed to incubate for 1 hour at 37 degrees Celsius. After incubation, each well was washed twice with PBS, column broken out, and placed into a Hidex gamma counter to assess the amount of radioactivity in each well. The percentage of bound activity was calculated based on a calibrated standard.

### 5. Surface Plasmon Resonance (SPR) Experiments

All SPR experiments were carried out using a Biacore T200 Biosensor instrument (GE Health Life Sciences, Piscataway, NJ). CM5 sensor chips, amine coupling reagents, and running buffers used in the binding analysis were obtained from Cytivalifesciences (Marlborough, MA).

### a. Immobilization of CD206

Recombinant human CD206 was purchased from R&D systems (Catalog #2534-MR-050). CD206 protein was immobilized onto a CM5 sensor chip surface via standard amine coupling chemistry, using HBS-P+ running buffer (10 mM HEPES, [pH 7.4], 150 mM NaCl, and 0.05% Surfactant 20) at 25° C., as per manufacturer recommendation (Cytiva, Marlborough, MA). Briefly, carboxyl groups on the sensor chip flow cell surface (flow cell 2) were activated by injecting an equal mixture of 200 mM N-hydroxysuccinimide (NHS) and 50 mM 1-ethyl-3-(3-dimethylaminopropyl) carbodiimide hydrochloride) (EDC) at a flow rate of 10 μl/min. CD206 (25 μg/mL) was then diluted in 10 mM sodium acetate buffer pH 5.0 and injected over the surface at a flow rate of 10 μl/min until a level of 5000 resonance units (RU) was reached. This was followed by a 7 min injection of 1 M ethanolamine (pH 8) to block excessive reactive esters and wash away any non-covalently bound CD206. A blank flow cell surface (flow cell 1) was similarly activated with no protein injection, and used as a reference to subtract non-specific binding and bulk refractive index.

### b. CD206 Peptide Binding Analysis

Peptide stock solutions were made as follows: lyophilized RP-832c peptide with DOTA was dissolved in molecular grade water, and the MART-1 peptide with DOTA was dissolved in a 1:5 ratio of 10 mM sodium acetate, and molecular grade water following manufacturer's protocol. The peptides were subsequently diluted in the HBS-P+ running buffer.

For the kinetic/affinity binding analysis, a series dilution of each peptide (in running buffer), ranging in concentrations from 0.5-10 μM, with at least one duplicate, were subsequently injected over the Blank (flow cell 1) and CD206 (flow cell 2) surfaces at 50 μL/min for a 60 second association time followed by a 180-600 second dissociation time. After each injection of the sensor chip, the surface was regenerated with a 30 second injection of 1M NaCl. Similarly, empty buffer injections were included as the first 5 cycles and then after every 5 cycles to save as blank controls. The binding analyses were further validated using duplicate injections of 0.5-10 μM concentrations of each peptide (two-times), injected using the same conditions.

### c. Data Processing, and Curve Fitting

The data processing for kinetic and affinity for CD206 and DOTA 832c binding interaction were performed using Biacore T200 Evaluation Software v2.0. All the binding sensorgrams were collected at 25° C. prior to curve fitting. The final data obtained which fit into a Langmuir 1:1 fit, were double-referenced using flow cell 1 and buffer blank subtractions.

### 6. Cell Culture and Animal Models

4T1 cells, a mouse breast cancer cell line (ATCC), and CT26 cells, a mouse colon carcinoma cell line (ATCC), were cultured in RPMI-1640 medium (ATCC) with 10 percent fetal bovine serum and 0.1 percent gentamicin at 37 degrees Celsius with 5 percent CO₂. Wild-type BALB/c mice (Charles River) were used for syngeneic 4T1 and CT26 tumor models and were subcutaneously injected in the shoulder with 200,000 4T1 cells and 2×10⁶ CT26 cells (with Matrigel). Tumors were allowed to grow for 10 days.

### 7. PET Imaging of [⁶⁸Ga]RP832c and [⁶⁸Ga]MART-1

Dynamic imaging studies were performed with a GNEXT small animal PET/CT (SOFIE). The mice were injected with [⁶⁸Ga]MART-1 and [⁶⁸Ga]RP832c four hours apart to allow time for decay from the first injections. The mice bearing CT26 tumors were injected with approximately 100 μCi (9-10 μg) of [⁶⁸Ga]MART-1 and 15 μCi of [⁶⁸Ga]832c via retro-orbital injection and then immediately underwent imaging. After 45 minutes, a high-resolution CT scan was obtained for anatomical reference. The same study was repeated in mice bearing 4T1 tumors with approximately 80 μCi (6-7 μg) of [⁶⁸Ga]MART-1 and 70 μCi (5-6 μg) of [⁶⁸Ga]832c via retro-orbital injection. After 1 hour, a high-resolution CT scan was obtained for anatomical reference. During imaging, mice were anesthetized with 2.5 percent isoflurane/oxygen. The PET data was reconstructed into 5-minute time frames.

Regions of interest (ROI) were drawn based on the CT anatomical guidelines. The mean standardized uptake value (SUV) was quantified using VivoQuant software with the equation:

$$SUV = \frac{C}{dose / weight}$$

Where C is the activity concentration in tissue, dose is the injected dose, and weight is the body weight of the mouse. Statistical analysis was conducted with Graphpad Prism software.

### 8. Biodistribution of [⁶⁸Ga]RP832c

Following the [⁶⁸Ga]832c imaging studies, animals were euthanized, and organs of interest removed and assessed for radioactivity. For all biodistribution studies, specific uptake for each tissue was measured with background and decay correction and expressed as percent injected dose per gram of tissue (% ID/g) as calculated by normalization to the total activity injected.

### 9. Immunohistochemistry

4T1 and CT26 tumors were collected following PET imaging on day 10. Tumors were cut at the largest cross section corresponding to in vivo imaging plane, fixed in 10% formalin, and transferred to 70% ethanol for immunohistochemistry. Each tumor was stained with hematoxylin and eosin (H&E) and 1:200 anti-CD206 (Catalog #: 60143-1-Ig, Proteintech Group). Slides were then incubated with Mouse/Rabbit IgG VisUCyte HRP Polymer antibody (Catalog #: VC002-050, R&D Systems) and counterstained with hematoxylin. Finally, slides were imaged with the EVOS M7000 20× objective (Thermo Fisher Scientific, Waltham, MA).

### B. RESULTS

### 1. Synthesis, Radiochemistry, and In Vitro Stability

FIG. 1 illustrates the radiochemical scheme of our PET CD206 targeting agent, [⁶⁸Ga]RP832c. ⁶⁸Ga was selected as an initial imaging isotope due to its half-life of 68 minutes and availability through generators, making it translatable to human studies. MART-1 was chosen as a nonbinding control and utilized the same radiolabeling conditions for the incorporation of ⁶⁸Ga. Quality control measures were taken through the use of radio-HPLC to confirm the radiochemical yield. Average radiochemical yields were greater than 99 percent for both [⁶⁸Ga]RP832c and [⁶⁸Ga]MART-1 (supplemental data). The radiolabeled compounds were obtained with high radiochemical purity (greater than 95% radiochemical yield), suitable for direct use in preclinical applications without further purification. FIG. 1 illustrates the radiosynthesis of [⁶⁸Ga]RP832c.

In vitro stability studies for both [⁶⁸Ga]RP832c and [⁶⁸Ga]MART-1 were conducted by measuring the radiochemical purity at incubation intervals in mouse serum and PBS at 37 degrees Celsius. At three hours, HPLC results demonstrated that ⁶⁸Ga remained complexed in both mouse serum and PBS (less than one percent free ⁶⁸Ga). Peptide degradation to approximately 65.48 and 63.78 percent radiochemical purity at three hours was apparent in the mouse serum studies for RP-832c and MART-1, respectively, but both remained intact in the PBS for up to three hours (supplemental data).

### 2. CD206 Binding Affinity of [⁶⁸Ga]RP832c

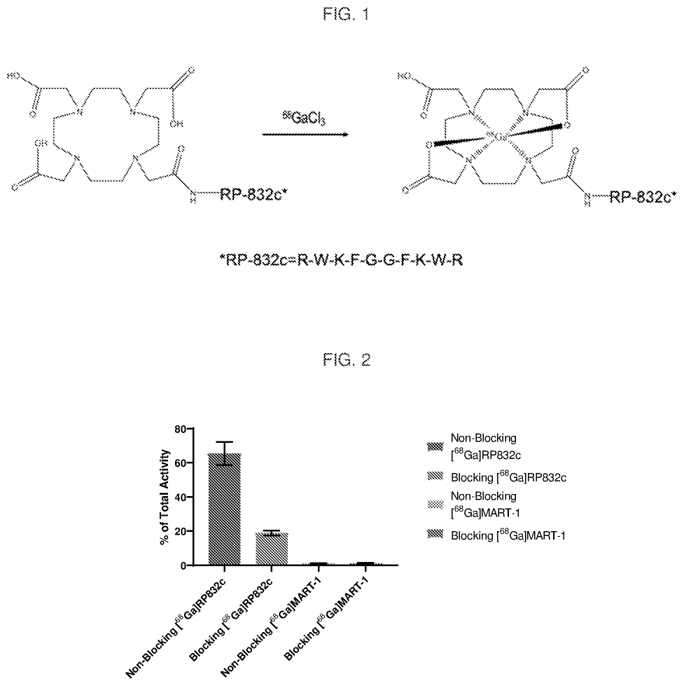

FIG. 2 demonstrates binding studies for [⁶⁸Ga]RP832c and [⁶⁸Ga]MART-1 to mouse CD206 protein. The experiment was divided into 2 separate groups for each tracer: The non-blocking group consisted of a 50 nM labeled peptide solution and PBS (equal volume), while the blocking group consisted of a 50 nM labeled peptide solution and a 5 μM unlabeled peptide blocking solution (equal volume). The results demonstrated high binding of [⁶⁸Ga]RP832c to mouse CD206 protein (average percentage of binding 65.39±6.82%) and that binding of the tracer was blocked significantly when incubated with the blocking solution (average percentage of binding 18.80±1.37%). Moreover, the percentage of binding for [⁶⁸Ga]MART-1, the control tracer, was not significant (average percentage of binding 0.94±0.13%). In addition, there was no statistically significant difference between the non-blocking and blocking groups for the [⁶⁸Ga]MART-1 studies (average percentage of binding 0.94±0.13% versus 0.87±0.56%). Taken together, this data indicates a significant binding of [⁶⁸Ga]RP832c to mouse CD206 and that MART-1 is a suitable control for continued studies due to its lack of binding. FIG. 2: Percentage of binding for [⁶⁸Ga]RP832c and [⁶⁸Ga]MART-1 to mouse CD206 protein. Non-blocking groups consisted of a 50 nM labeled peptide solution and PBS. Blocking groups consisted of a 50 nM labeled peptide solution and a 5 μM unlabeled peptide blocking solution.

### 3. Surface Plasmon Resonance

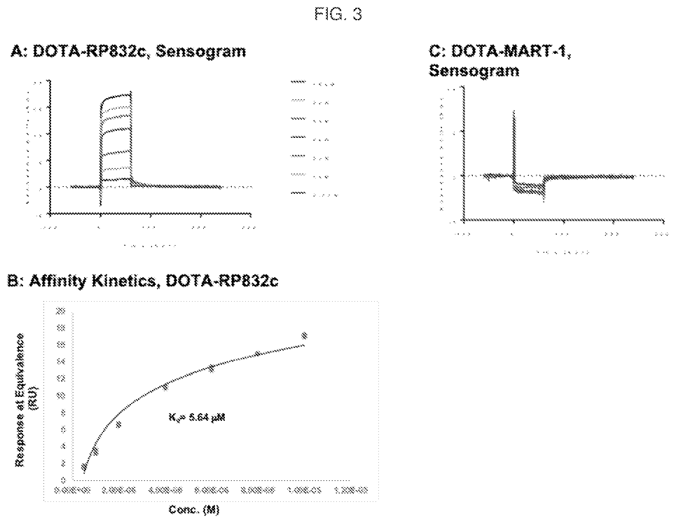

To further validate the binding affinity of RP832 and MART-1 to CD206, Surface Plasmon Resonance experiments were conducted (FIGS. 3A-3C). Each peptide was diluted in running buffer with varying concentrations (0.5 uM-10 uM). An affinity kinetics curve fitting of CD206 and DOTA-RP832c produced a calculated dissociation constant (K_{d}) of 5.64 uM. The binding sensorgram of CD206 and DOTA-MART-1 showed no binding in all concentrations (0.5 uM-10 uM).

FIGS. 3A-3C. A. Binding sensorgram of CD206 and DOTA-832c peptide diluted in a series of running buffer (dilutions ranging in concentrations from 0.5 uM-10 uM). B. Affinity kinetics curve fitting of CD206 and DOTA-832c. Calculated Dissociation constant (K_{d})=5.64 uM. C. Binding sensorgram of CD206-DOTA MART-1 shows no binding in all concentrations (0.5 uM-10 uM).

### 4. PET/CT Imaging Biodistribution

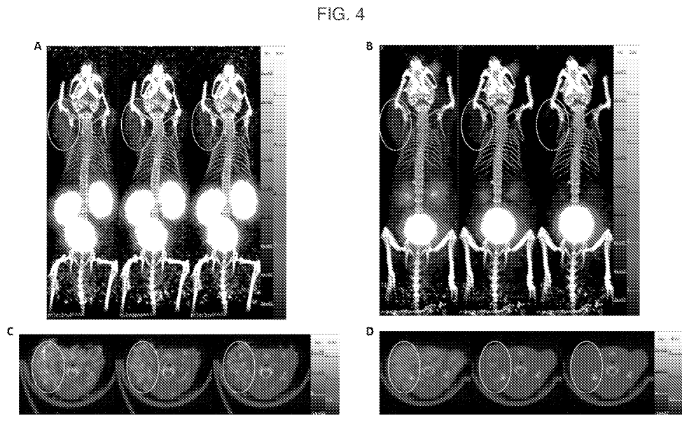

Dynamic PET imaging studies were conducted in BALB/c mice bearing syngeneic CT26 tumors. [⁶⁸Ga]MART-1, a tracer utilized to serve as a control, was injected and imaged 4 hours prior to the injection and imaging of [⁶⁸Ga]832c, in the same mice. Dynamic PET data collected over 45 minutes demonstrated a positive tumor uptake with [⁶⁸Ga]832c showing higher uptake when compared to the control at later time-points (FIGS. 4A-4D). The axial view for the images showed retention of [⁶⁸Ga]832c in the tumor over time (average mean SUV 0.50 40 minutes post injection) while [⁶⁸Ga]MART-1 images demonstrated clearance from the tumor over time (average mean SUV 0.24 40 minutes post injection). The maximum intensity projections (MIPs) showed the highest signal in the kidneys and bladder for [⁶⁸Ga]RP832c and the bladder for [⁶⁸Ga]MART-1, suggesting renal clearance.

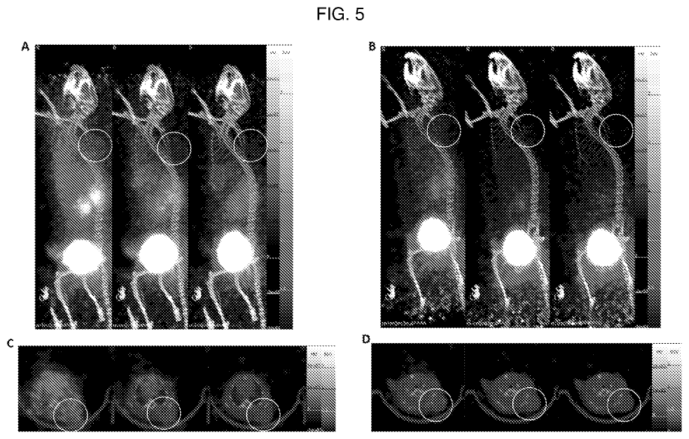

The same study was repeated in BALB/c mice bearing syngeneic 4T1 tumors (FIGS. 5A-5D). Dynamic PET data collected over 1 hour demonstrated a difference in uptake and overall distribution between the two tracers at earlier time-points. Tumor uptake was visualized in the axial images where [⁶⁸Ga]RP832c (average mean SUV 0.25 15 minutes post injection) uptake was significantly higher when compared to uptake of [⁶⁸Ga]MART-1 (Average mean SUV 0.07 15 minutes post injection). MIP images showed the highest signal to be in the bladder for both [⁶⁸Ga]RP832c and [⁶⁸Ga]MART-1.

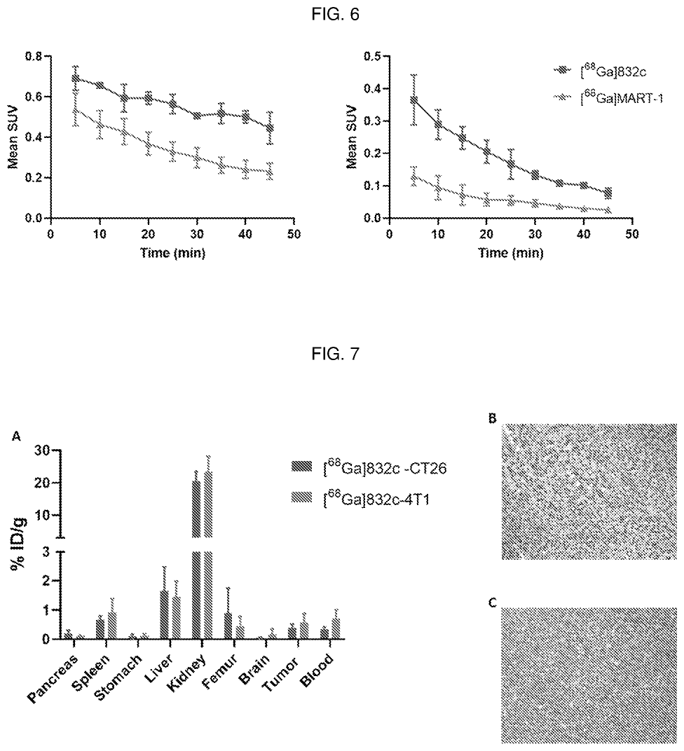

FIG. 6 shows time activity curves comparing the mean SUV over time of [⁶⁸Ga]RP832c and [⁶⁸Ga]MART-1 in CT26 and 4T1 tumor models. Mean SUV values for the CT26 study data demonstrated a greater difference between [⁶⁸Ga]RP832c and [⁶⁸Ga]MART-1 at later time points. [⁶⁸Ga]RP832c uptake in the tumor was statistically higher than [⁶⁸Ga]MART-1 15-45 minutes post injection. The 4T1 study data demonstrated a greater difference between the two tracers at earlier time-points. Statistical significance between the two tracers was shown during 0-45 minutes post injection.

### 5. Biodistribution and Immunohistochemistry

Following PET/CT imaging of [⁶⁸Ga]RP832c, each tracer was further assessed ex vivo by collecting organs of interest. The uptake was apparent in tumor and CD206 expressing organs such as liver and spleen in both 4T1 and CT26 tumor models (FIGS. 7A-7C). The average percent injected dose per gram of organ for the tumor, spleen, and liver was 0.57, 0.91, and 1.46 in the 4T1 model and 0.40, 0.67, and 1.66 in the CT26 model, respectively. The tumors were saved after biodistribution in order to conduct immunohistochemistry for the assessment of CD206 expression in each tumor (FIGS. 7A-7C).

### C. DISCUSSION

Tumor-associated macrophages (TAMs) play various roles in cancer biology and are integral to the relationship between immune system response and tumor progression. As a result, several TAMs targeted therapies have been developed in the preclinical and clinical settings.¹⁴ RP-832c is a peptide that exhibits therapeutic properties due to its ability to specifically target the CD206 receptor expressed on M2 macrophages and induce conformational changes which lead to a repolarization of the macrophage to the M1 phenotype upon binding to the receptor.⁹

RP832c recognizes both murine and human CD206 and was conjugated with the bifunctional chelator 1,4,7,10-tetraazacyclododecane-1,4,7,10-tetraacetic acid (DOTA) and radiolabeled with ⁶⁸Ga. Stability studies in PBS and mouse serum demonstrated that ⁶⁸Ga remained complexed to DOTA up to three hours confirming the high stability of ⁶⁸Ga-DOTA moieties. Degradation to approximately 65 percent radiochemical purity of the peptide was shown when incubated in mouse serum but is still above 50 percent and far exceeds the time of imaging and biodistribution studies that were conducted. The CD206 protein plate binding assay confirmed binding of [⁶⁸Ga]RP832c to mouse CD206 as well as the choice of [⁶⁸Ga]MART-1 as a nonbinding control due to its significantly lower percentage of binding.

In mice, [⁶⁸Ga]RP832c accumulated in CT26 and 4T1 tumors as well as CD206 expressing organs such as the liver and the spleen. Dynamic PET/CT data demonstrated that statistical significance between [⁶⁸Ga]RP832c and [⁶⁸Ga]MART-1 in the tumor at specific time-points. Partial signal decrease may be attributed to [⁶⁸Ga]RP832c cycling on and off with red blood cells which are the natural carriers for RP832c but further studies are required to confirm this. 15Imaging macrophages using CD206 receptor targeting has been explored previously. The sole FDA approved tracer for imaging macrophages is the SPECT agent ^{99m}Tc-Tilmanocept which is used in guiding sentinel node biopsy in several different types of tumors. The structure of Tilmanocept consists of mannose moieties which bind to CD206.¹⁴ The targeting of Tilmanocept relies on the use of the natural ligand mannose which binds the CD206 receptor but also multiple other mannose-binding proteins which makes it somewhat less specific for the identification of CD206 expressing macrophages.¹⁶

Fairly recently, a ⁶⁸Ga labeled single-domain antibody fragment has been used for the PET imaging of macrophages ([⁶⁸Ga]Ga-NOTA-anti-MMR-sdAb).¹⁶ This tracer shows promise for specifically targeting CD206, but the use of peptides provides advantageous characteristics when compared to antibodies and fragments, such as less toxicity and lower immunogenic potential.

D. CONCLUSION

We developed and evaluated a novel CD206-targeted PET probe in syngeneic murine models of cancer. [⁶⁸Ga]RP832c showed a strong binding to murine CD206, a high stability with respect to complexation, and illustrated uptake in tumor and CD206 expressing organs such as the spleen and the liver. [⁶⁸Ga]RP832c represents a promising candidate for macrophage imaging.

### E. REFERENCES

1. Zhou, J.; Tang, Z.; Gao, S.; Li, C.; Feng, Y.; Zhou, X., Tumor-Associated Macrophages: Recent Insights and Therapies. Front Oncol 2020, 10, 188.
2. Lin, Y.; Xu, J.; Lan, H., Tumor-associated macrophages in tumor metastasis: biological roles and clinical therapeutic applications. J Hematol Oncol 2019, 12 (1), 76.
3. Petty, A. J.; Yang, Y., Tumor-associated macrophages: implications in cancer immunotherapy. Immunotherapy 2017, 9 (3), 289-302.
4. Wynn, T. A.; Chawla, A.; Pollard, J. W., Macrophage biology in development, homeostasis and disease. Nature 2013, 496 (7446), 445-55.
5. Chen, Y.; Song, Y.; Du, W.; Gong, L.; Chang, H.; Zou, Z., Tumor-associated macrophages: an accomplice in solid tumor progression. Journal of Biomedical Science 2019, 26 (1), 78.
6. Qian, B. Z.; Pollard, J. W., Macrophage diversity enhances tumor progression and metastasis. Cell 2010, 141 (1), 39-51.
7. Tsuchiya, K.; Suzuki, Y.; Yoshimura, K.; Yasui, H.; Karayama, M.; Hozumi, H.; Furuhashi, K.; Enomoto, N.; Fujisawa, T.; Nakamura, Y.; Inui, N.; Yokomura, K.; Suda, T., Macrophage Mannose Receptor CD206 Predicts Prognosis in Community-acquired Pneumonia. Scientific Reports 2019, 9 (1), 18750.
8. Suzuki, Y.; Shirai, M.; Asada, K.; Yasui, H.; Karayama, M.; Hozumi, H.; Furuhashi, K.; Enomoto, N.; Fujisawa, T.; Nakamura, Y.; Inui, N.; Shirai, T.; Hayakawa, H.; Suda, T., Macrophage mannose receptor, CD206, predict prognosis in patients with pulmonary tuberculosis. Scientific Reports 2018, 8 (1), 13129.
9. Ghebremedhin, A.; Salam, A. B.; Adu-Addai, B.; Noonan, S.; Stratton, R.; Ahmed, M. S. U.; Khantwal, C.; Martin, G. R.; Lin, H.; Andrews, C.; Karanam, B.; Rudloff, U.; Lopez, H.; Jaynes, J.; Yates, C., A Novel CD206 Targeting Peptide Inhibits Bleomycin Induced Pulmonary Fibrosis in Mice. bioRxiv 2020.
10. Jaynes, J. M.; Sable, R.; Ronzetti, M.; Bautista, W.; Knotts, Z.; Abisoye-Ogunniyan, A.; Li, D.; Calvo, R.; Dashnyam, M.; Singh, A.; Guerin, T.; White, J.; Ravichandran, S.; Kumar, P.; Talsania, K.; Chen, V.; Ghebremedhin, A.; Karanam, B.; Bin Salam, A.; Amin, R.; Odzorig, T.; Aiken, T.; Nguyen, V.; Bian, Y.; Zarif, J. C.; de Groot, A. E.; Mehta, M.; Fan, L.; Hu, X.; Simeonov, A.; Pate, N.; Abu-Asab, M.; Ferrer, M.; Southall, N.; Ock, C. Y.; Zhao, Y.; Lopez, H.; Kozlov, S.; de Val, N.; Yates, C. C.; Baljinnyam, B.; Marugan, J.; Rudloff, U., Mannose receptor (CD206) activation in tumor-associated macrophages enhances adaptive and innate antitumor immune responses. Sci Transl Med 2020, 12 (530).
11. Wang, Y.; Liu, S.; Yang, Z.; Algazi, A. P.; Lomeli, S. H.; Wang, Y.; Othus, M.; Hong, A.; Wang, X.; Randolph, C. E.; Jones, A. M.; Bosenberg, M. W.; Byrum, S. D.; Tackett, A. J.; Lopez, H.; Yates, C.; Solit, D. B.; Ribas, A.; Piva, M.; Moriceau, G.; Lo, R. S., Anti-PD-1/L1 lead-in before MAPK inhibitor combination maximizes antitumor immunity and efficacy. Cancer Cell 2021, 39 (10), 1375-1387.e6.
12. Vaquero, J. J.; Kinahan, P., Positron Emission Tomography: Current Challenges and Opportunities for Technological Advances in Clinical and Preclinical Imaging Systems. Annu Rev Biomed Eng 2015, 17, 385-414.
13. Mueller, D.; Breeman, W. A.; Klette, I.; Gottschaldt, M.; Odparlik, A.; Baehre, M.; Tworowska, I.; Schultz, M. K., Radiolabeling of DOTA-like conjugated peptides with generator-produced (68)Ga and using NaCl-based cationic elution method. Nat Protoc 2016, 11 (6), 1057-66.
14. Parker, C. C.; Lapi, S. E., Positron Emission Tomography Imaging of Macrophages in Cancer. Cancers 2021, 13 (8), 1921.
15. Oronsky, B.; Paulmurugan, R.; Foygel, K.; Scicinski, J.; Knox, S. J.; Peehl, D.; Zhao, H.; Ning, S.; Cabrales, P.; Summers, T. A.; Reid, T. R.; Fitch, W. L.; Kim, M. M.; Trepel, J. B.; Lee, M.-J.; Kesari, S.; Abrouk, N. D.; Day, R. M.; Oronsky, A.; Ray, C. M.; Carterg, C. A., RRx-001: a systemically non-toxic M2-to-M1 macrophage stimulating and prosensitizing agent in Phase II clinical trials. Expert Opinion on Investigational Drugs 2017, 26 (1), 109-119.
16. Xavier, C.; Blykers, A.; Laoui, D.; Bolli, E.; Vaneyken, I.; Bridoux, J.; Baudhuin, H.; Raes, G.; Everaert, H.; Movahedi, K.; Van Ginderachter, J. A.; Devoogdt, N.; Caveliers, V.; Lahoutte, T.; Keyaerts, M., Clinical Translation of [68Ga]Ga-NOTA-anti-MMR-sdAb for PET/CT Imaging of Protumorigenic Macrophages. Molecular Imaging and Biology 2019, 21 (5), 898-906.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A conjugate comprising:
a CD206 binding peptide conjugated to a label or therapeutic agent.

2. The conjugate according to claim 1, wherein the peptide comprises:
a) a peptide sequence selected from RWKFGGFKWR (RP832C) (SEQ ID NO: 96); FWKRFVRKWR (RP837) (SEQ ID NO: 97); FKWRGGRWKF (RP837C) (SEQ ID NO: 98); FWKRGGRKWF (RP837A) (SEQ ID NO: 99); FWKRFV (RP837N) (SEQ ID NO: 100); FWKKFVKKWK (RP841) (SEQ ID NO:101); GDRGIKGHRGF (RP842) (SEQ ID NO: 102); EKLSAFRNFF (RP843) (SEQ ID NO: 103); FYPDFFKKFF (RP844) (SEQ ID NO: 104); FFRHFATHLD (RP845) (SEQ ID NO: 105); LYKKIIKKLL (RP846) (SEQ ID NO: 106); WWHHWWHHWH (RP847) (SEQ ID NO: 107); WWRHWWHRWR (RP848) (SEQ ID NO: 108); WWKHWWHKWK (RP849) (SEQ ID NO: 109); FVRKWR (RP837C1) (SEQ ID NO: 110); FFRKSKEKIG (RP853) (SEQ ID NO: 111); FAOOFAOOFO (RP850) (SEQ ID NO: 112); or
b) a sequence having one or two amino acid substitutions relative to the sequence defined in a).

3. The conjugate according to any of the preceding claims, wherein the peptide has a sequence selected from: RWKFGGFKWR (RP832C) (SEQ ID NO: 96); FWKRFVRKWR (RP837) (SEQ ID NO: 97); FKWRGGRWKF (RP837C) (SEQ ID NO: 98); FWKRGGRKWF (RP837A) (SEQ ID NO: 99); FWKRFV (RP837N) (SEQ ID NO: 100); FWKKFVKKWK (RP841) (SEQ ID NO:101); GDRGIKGHRGF (RP842) (SEQ ID NO: 102); EKLSAFRNFF (RP843) (SEQ ID NO: 103); FYPDFFKKFF (RP844) (SEQ ID NO: 104); FFRHFATHLD (RP845) (SEQ ID NO: 105); LYKKIIKKLL (RP846) (SEQ ID NO: 106); WWHHWWHHWH (RP847) (SEQ ID NO: 107); WWRHWWHRWR (RP848) (SEQ ID NO: 108); WWKHWWHKWK (RP849) (SEQ ID NO: 109); FVRKWR (RP837C1) (SEQ ID NO: 110); FFRKSKEKIG (RP853) (SEQ ID NO: 111); FAOOFAOOFO (RP850) (SEQ ID NO: 112).

4. The conjugate according to claim 3, wherein the peptide has the sequence RWKFGGFKWR (RP832C) (SEQ ID NO: 96).

5. The conjugate according to claim 1, wherein the peptide comprises a Class II peptide.

6. The conjugate according to claim 5, wherein the peptide comprises:
a) a peptide selected from RP124, RP132, RP134, RP142, RP147, RP151, RP166-RP172, RP175, RP177, RP182, RP183, RP185, RP186, RP 424, RP190, RP194, RP198, RP199-RP202, RP204, RP206, RP207, RP209, RP210, RP212-RP216, RP218, RP219, RP425, RP225, RP227, RP233-RP239, RP398, RP241-RP247, RP250-RP256, RP426, RP427, RP285, and RP387; or
b) a sequence having one or two amino acid substitutions relative to the sequence defined in a).

7. The conjugate according to any of the preceding claims, wherein the peptide is selected from: RP124, RP132, RP134, RP142, RP147, RP151, RP166-RP172, RP175, RP177, RP182, RP183, RP185, RP186, RP 424, RP190, RP194, RP198, RP199-RP202, RP204, RP206, RP207, RP209, RP210, RP212-RP216, RP218, RP219, RP425, RP225, RP227, RP233-RP239, RP398, RP241-RP247, RP250-RP256, RP426, RP427, RP285, and RP387.

8. The conjugate according to claim 7, wherein the peptide is RP-182.

9. The conjugate according to claim 7, wherein the peptide is RP-185.

10. The conjugate according to any of the preceding claims, wherein the peptide is conjugated to a label.

11. The conjugate according to claim 10, wherein the label is selected from the group consisting of a radionuclide, a radiological contrast agent, a paramagnetic ion, a metal, a biological tag, a fluorescent label, a chemiluminescent label, an ultrasound contrast agent and a photoactive agent.

12. The conjugate according to claim 11, wherein the label comprise a radionuclide.

13. The conjugate according to claim 7, wherein the radionuclide is selected from the group consisting of ¹¹⁰In, ¹¹¹In, ¹⁷⁷Lu, ¹⁸F, ⁵²Fe, ⁶²Cu, ⁶⁴Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

14. The conjugate according to any of claims 1 to 9, wherein the peptide is conjugated to a therapeutic agent.

15. The conjugate according to claim 9, wherein the therapeutic agent is selected from the group consisting of cytotoxic agents, anti-angiogenic agents, pro-apoptotic agents, antibiotics, hormones, hormone antagonists, chemokines, drugs, prodrugs, toxins, enzymes, antimitotics, antikinases, alkylating agents, antimetabolites and alkaloids.

16. The conjugate according to claim 15, wherein the cytotoxic agent is a cytotoxic radionuclide.

17. A pharmaceutical composition, comprising the conjugate of any of the preceding claims and a pharmaceutically acceptable carrier.

18. A method of detecting whether a tumor associated macrophage is present in a sample, the method comprising:
combining the sample with a conjugate a conjugate comprising a CD206 binding peptide conjugated to a label to produce a labeled sample; and
assessing the labeled sample for the presence of the label to detect whether the tumor associated macrophage is present in the sample.

19. The method according to claim 18, wherein the macrophages are associated with a tumor.

20. The method according to claim 19, wherein the tumor is a cancer selected from the group consisting of squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, and head and neck cancer.

21. The method according to any of claims 18 to 20, wherein assessing comprising employing an imaging technique.

22. The method according to claim 21, wherein the imaging technique is selected from the group consisting of fluorescence, positron emission tomography (PET), magnetic resonance imaging (MRI), single photon emission computed tomography (SPECT/CT), intravital laser scanning microscopy, endoscopy, and radiographic imaging.

23. The method according to any of claims 18 to 22, wherein the tumor associated macrophage is an M2 macrophage.

24. The method according to any of claims 18 to 23, wherein the sample is an in vivo sample.

25. The method according to any of claims 18 to 23, wherein the sample is an in vitro sample.

26. The method according to any of claims 18 to 23, wherein the method is used to monitor the location of tumor associated macrophages.

27. A method of detecting whether fibrosis is present in a sample, the method comprising:
combining the sample with a conjugate a conjugate comprising a CD206 binding peptide conjugated to a label to produce a labeled sample; and
assessing the labeled sample for the presence of the label to detect whether the fibrosis is present in the sample.

28. The method according to claim 27, wherein assessing comprising employing an imaging technique.

29. The method according to claim 28, wherein the imaging technique is selected from the group consisting of fluorescence, positron emission tomography (PET), magnetic resonance imaging (MRI), single photon emission computed tomography (SPECT/CT), intravital laser scanning microscopy, endoscopy, and radiographic imaging.

30. The method according to any of claims 27 to 29, wherein the sample is an in vivo sample.

31. The method according to any of claims 27 to 29, wherein the sample is an in vitro sample.

32. A method for treating a subject for a condition, the method comprising:
administering to the subject an effective amount of a conjugate comprising a CD206 binding peptide conjugated to a therapeutic agent to treat the subject for the condition.

33. The method according to claim 32, wherein the condition is cancer.

32. ethod according to claim 32, wherein the condition is fibrosis.

35. The method according to claim 34, wherein the fibrosis is pulmonary fibrosis, liver fibrosis, renal fibrosis, heart fibrosis and scleroderma.
